(19) United States
(12) Patent Application Publication
(10) Pub. No.: US 2006/0018937 A1
(43) Pub. Date: Jan. 26, 2006

(54) Steroid kit and foamable composition and uses thereof

(75) Inventors: Doron Friedman, Karmei Yosef (IL); Alex Besonov, Rehovet (IL); Dov Tamarkin, Maccabim (IL); Meir Eini, Ness Ziona (IL)

Correspondence Address: WILMER CUTLER PICKERING HALE AND DORR LLP, 60 STATE STREET, BOSTON, MA, 02109, US

(73) Assignee: Foamix Ltd.

(21) Appl. No.: 11/114,410

(22) Filed: Apr. 26, 2005

Related U.S. Application Data
(63) Continuation-in-part of application No. 10/911,367, filed on Aug. 4, 2004.
(63) Continuation-in-part of application No. 10/532,618.
(60) Provisional application No. 60/492,385, filed on Aug. 4, 2003.
(60) Provisional application No. 60/429,546, filed on Nov. 29, 2002.

(30) Foreign Application Priority Data
Oct. 24, 2003 (WO) PCT/IB03/05527
Oct. 25, 2002 (IL) 152486

(51) Int. Cl. A61K 8/02

(52) U.S. Cl. 424/401

(57) ABSTRACT

A composition and therapeutic kit including an aerosol packaging assembly including a container accommodating a pressurized product and an outlet capable of releasing a foamable composition, including a steroid as a foam. The pressurized product includes a foamable composition including: a container accommodating a pressurized product; and an outlet capable of releasing the pressurized product as a foam; wherein the pressurized product comprises a foamable composition including: i. a steroid; ii. at least one organic carrier selected from the group consisting of a hydrophobic organic carrier, a polar solvent, an emollient and mixtures thereof, at a concentration of about 2% to about 50% by weight; iii. a surface-active agent; iv. about 0.01% to about 5% by weight of at least one polymeric additive selected from the group consisting of a bioadhesive agent, a gelling agent, a film forming agent and a phase change agent; v. water; and vi. liquefied or compressed gas propellant at a concentration of about 3% to about 25% by weight of the total composition. The composition further may include a therapeutically active foam adjuvant, selected from the group consisting of a fatty alcohol, a fatty acid, a hydroxyl fatty acid; and mixtures thereof.

### CROSS REFERENCE TO RELATED APPLICATIONS

This application is a continuation-in-part application of co-pending International Patent Application No. IB03/005527, designating the United States and filed on Oct. 24, 2003, which claims the benefit of priority under 35 U.S.C. §119(e) to U.S. Patent Application Ser. No. 60/492,546, filed on Nov. 29, 2002, both entitled “Cosmetic and Pharmaceutical Foam,” and which claims the benefit of priority under 35 USC §119(a) to Israeli Patent Application. No. 152486, filed Oct. 25, 2002, all of which are hereby incorporated in their entirety by reference.

This application is a continuation-in-part application of co-pending U.S. patent application Ser. No. 10/911,367, filed on Aug. 4, 2004, which claims the benefit of priority under 35 U.S.C. §119(e) to U.S. Patent Application Ser. No. 60/492,385, filed on Aug. 4, 2003, both entitled “Foam Carrier Containing Amphiphilic Copolymer Gelling Agent” and both hereby incorporated in their entirety by reference.

### BACKGROUND OF THE INVENTION

Steroids are available in topical dosage form. Compositions containing steroids for topical treatment of dermatologic disorders are available primarily in cream, lotion gel and ointment forms. While semi-solid compositions, such as creams, lotions, gels and ointments are commonly used by consumers, new forms are desirable, in order to achieve better control of the application, while maintaining or bestowing the skin beneficial properties of such products. Thus, the development of new compositions having breakable foam consistency when released from a container and liquid properties when applied onto the skin are advantageous.

Foams and, in particular, foam emulsions are complicated systems which do not form under all circumstances. Slight shifts in foam emulsion composition, such as by the addition of active ingredients, may destabilize the foamable composition during storage, and/or impair the quality of the foam that is produced upon release from the aerosol container.

U.S. Pat. No. 6,126,920 discloses treatment of various skin diseases, and in particular, scalp psoriasis, using a foamable pharmaceutical composition containing a corticosteroid active substance, an aliphatic alcohol, water, a fatty alcohol, a surface-active agent, a propellant and a buffering agent. The foamable composition contains 40-90% w/w composition of an aliphatic alcohol. U.S. Pat. No. 6,126,920 is typical of many compositions that use aliphatic alcohols in the foam composition. The alcohol promotes fast drying and thereby attempts to address the sticky feeling left by many topical formulations after application; however, alcohols, and in particular the methyl, ethyl and isopropyl alcohols preferred in the '920 patent, are defatting agents and may cause skin to become dry and cracked. U.S. Pat. No. 6,730,288 teaches a pharmaceutical foam composition including (a) an active ingredient; (b) an occlusive agent; (c) an aqueous solvent; and (d) an organic cosolvent; wherein the active ingredient is insoluble in water and insoluble in both water and the occlusive agent; and wherein there is enough occlusive agent to form an occlusive layer on the skin.

A few dermatological foam products are available on the market.

Olux™ Foam, produced by Connetics, Inc., contains clobetasol propionate. Each gram of Olux™ Foam contains 0.5 mg clobetasol propionate, USP, in a thermolabile foam, which consists of ethanol (60%), purified water, propylene glycol, cetyl alcohol, stearyl alcohol, polysorbate 60, citric acid, and potassium citrate. It is dispensed from an aluminum can pressurized with a hydrocarbon propellant (propane/butane). Luxiq™ is another corticostroid foam medication, containing 1.2 mg betamethasone valerate per gram, in a vehicle, comprising ethanol (60.4%), purified water, propylene glycol, cetyl alcohol, stearyl alcohol, polysorbate 60, citric acid, and potassium citrate, and pressurized with a hydrocarbon propellant. Cortifoam™, a 10 wt % hydrocortisone acetate rectal foam, is produced by Schwartz Pharma GmbH. Non medicinal ingredients of Cortifoam™ include cetyl alcohol, ethoxylated stearyl alcohol, methylparaben, polyoxyethylene-10 stearyl ether, propylene glycol, propylparaben, triethanolamine, water, and inert propellants, isobutene, and propane.

Thus, low alcohol content foam compositions for topical treatment containing higher concentrations of oils are not currently commercially available. Foam compositions that are robust and suitable for inclusion of a wide range of active ingredients are desired.

### SUMMARY OF THE INVENTION

The present invention provides a therapeutic kit including a foamable steroid composition. The kit includes an aerosol packaging assembly having a container accommodating a pressurized product and an outlet capable of releasing the pressurized product as a foam.

The therapeutic kit includes an aerosol packaging assembly including (a) a container accommodating a pressurized product and (b) outlet capable of releasing the pressurized product as a foam; wherein the pressurized product contains a foamable composition including: (i) a steroid; (ii) at least one organic carrier selected from the group consisting of a hydrophobic organic carrier, an organic polar solvent, an emollient and mixtures thereof, at a concentration of about 2% to about 50% by weight; (iii) a surface-active agent; (iv) about 0.01% to about 5% by weight of at least one polymeric additive selected from the group consisting of a bioadhesive agent, a gelling agent, a film forming agent and a phase change agent; (v) water; and (vi) liquefied or compressed gas propellant at a concentration of about 3% to about 25% by weight of the total composition.

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butanol, iso-butanol, t-butanol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

In preferred embodiments, the composition further contains a therapeutically effective foam adjuvant to increase the foaming capacity of surfactants and/or to stabilize the foam. In one or more embodiments of the present invention, the foam adjuvant includes fatty alcohols having 15 or more carbons in their carbon chain, fatty acids having 16 or more carbons in their carbon chain, and combinations or mixtures thereof.

In one or more embodiments, a combination of a fatty acid and a fatty ester is employed.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

In one or more embodiments, at least a portion of the steroid is suspended in the composition, yet, in other embodiments, the steroid is dissolved in the composition.

In one or more embodiments, the foam composition is formulated as an oil-in-water emulsion or oil-in-water microemulsion.

The steroid according to the present invention is selected from the group consisting of:
(i) a steroid compound containing a cyclopenta[a]phenanthrene skeleton;
(ii) a steroid compound containing a cyclopenta[a]phenanthrene skeleton carrying one or more functional groups selected from halogens, alkyl groups, aryl groups, benzyl groups, carboxy groups and alkoxy groups;
(iii) a steroid compound selected from the families of (a) cardanolides, (b) bufanolides, (c) spirostans, (d) furostans, (e) steroid alkaloids, (f) steroid lactones, (g) oxo-steroids, (h) steroid-alcohols and (i) steroid-amines;
(iv) a steroid compound, where one or more of the cyclopenta[a]phenanthrene rings is contracted by loss of an unsubstituted methylene group;
(v) a steroid compound, where one or more of the cyclopenta[a]phenanthrene rings is expanded by inclusion of a methylene group;
(vi) a steroid compound containing a cyclopenta[a]phenanthrene skeleton and a carbocyclic or heterocyclic ring component fused to it;
(vii) a compound, wherein two or more steroid molecules are linked together covalently;
(viii) a compound selected from the group consisting of 5α-pregnane, 5^{β}-pregnane, 5α-cholane (allocholane), 5^{β}-cholane, 5α-cholestane, 5^{β}-cholestane, 5α-ergostane, 5^{β}-ergostane, 5α-campestane, 5^{β}-campestane, 5α-poriferastane, 5^{β}-poriferastane, 5α-stigmastane, 5^{β}-stigmastane, 5α-gorgostaneacrihellin, actodigin, alfacalcidol, aldosterone, androsterone, betamethasone, brassinolide, calcidiol, calciol, calcitriol, canrenone, clomegestone, cholesterol, cholic acid, corticosterone, cortisol, cortisol acetate, cortisone, cortisone acetate, cyproterone, deoxycorticosterone, dexamethasone, disogluside, ecdysone, ercalciol, ergosterol, estradiol, estriol, estrone, ethinylestradiol, fluazacort, fluocortin, fusidic acid, gestrinone, gonane, halometasone, hydrocortisone, lanosterol, lithocholic acid, mebolazine, medroxyprogesterone, meproscillarin, mespirenone, mestranol, naflocort, norenthisterone, norgesterone, norgestrel, oxandrolone, oxymetholone, pancuronium bromide, prednisolone, prednisone, progesterone, proscillardin, pseudotigogenin, roxibolone, sarsasapogenin, smilagenin, spironolactone, timobesone, testosterone, tigogenin triamcinolone, ursodeoxycholic acid;
(ix) an anti-inflammatory steroid;
(x) a steroid possessing immunomodulating and/or anti-inflammatory properties;
(xi) a steroid, selected from the group of low-potency anti-inflammatory steroids, medium potency anti-inflammatory steroids and high potency anti-inflammatory steroids;
(xii) an anti-inflammatory steroid, selected from the group consisting of hydrocortisone, hydrocortisone acetate, desonide, betamethasone valerate, clobetasone-17-butyrate, flucinonide, fluocinolone acetonide, alcometasone dipropionate, mometasone furoate, prednicarbate, triamcinolone acetonide, betamethasone-17-benzoate, methylprednisolone aceponate, betamethasone dipropionate, halcinonide, triamcinolone acetonide, halobetasol, clobetasol-17-propionate;
(xiii) a steroid that positively affects the McKenzie vasoconstrictor assay;
(xiv) a steroid hormone;
(xv) a steroid hormone, selected from the group consisting of an androgen, an estrogen and a progestogen;
(xvi) an androgen, selected from the group consisting of testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone (also termed prasterone and DHEA), androstenedione, androstanediol, androsterone, androstenolone, prasterone enantate, prasterone sodium sulfate, ormeloxifene, mesterolone, fluoxymesterone, methyltestosterone, gestrinone, delmadinone, delmadinone acetate, chlormadinone, chlormadinone acetate, danazol and testolactone;
(xvii) an estrogen selected from the group consisting of estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate and equilin;
(xviii) a progestogen, selected from the group consisting of progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlormadinone acetate, dienogest, drospirenone, lynestrenol, tybolone, cyproterone acetate, megestrol acetate, nomegestrol acetate;
(xix) an inhibitor of a steroid hormone;
(xx) an inhibitor of a steroid hormone selected from the group consisting of finasteride, dutasteride and spironolactone;
(xxi) a vitamin D;
(xxii) a steroid that exhibits qualitatively the biological activity of calciol;
(xxiii) a vitamin D selected from the group consisting of cholecalciferol, 25-hydroxycholecalciferol, 1α,25-dihydroxycholecalciferol, ergocalciferol, 1α,25-dihydroxyergocalciferol, 22,23-dihydroergocalciferol, 1,24,25-trihydroxycholecalciferol, previtamin D₃, tachysterol₃ (also termed tacalciol);
(xxiv) isovitamin D₃, dihydrotachysterol₃, (1S)-hydroxycalciol, (24R)-hydroxycalcidiol, 25-fluorocalciol, ercalcidiol, ertacalciol, (5E)-isocalciol, 22,23-dihydroercalciol, (24S)-methylcalciol, (5E)-(10S)-10,19-dihydroercalciol, (24S)-ethylcalciol and (22 E)-(24R)-ethyl-22,23-didehydrocalciol;
(xxv) a phytosteroid or a phytosterol;
(xxvi) a steroid derived or extracted from one of the families of phytosteroids, phytosterols, phytostanols, ecdysones, withanolids, sterines, steroid saponins and soflavonoids;
(xxvii) a steroid selected from the group consisting of alpha-sitosterol, beta-sitosterol, stigmastanol, campesterol, alpha-sitostanol, beta-sitostanol, stigmastanol, campestanol, avenosterol, brassicasterol, desmosterol, chalinosterol, beta-ecdysone, whithaferin A, beta-sitosterine, stigmasterine, campesterine, ergosterine, diosgenin, daidzein, glycitein, genistein, muristerone, poriferasterol, clionasterol, campestanol, and cycloartenol;
(xxviii) a plant oil or a plant extract, which contains a steroid;
(xxix) a plant oil or a plant extract, selected from the group consisting of nuts seeds, sprouted seeds and grains (such as alfalfa), St. Mary's thistle, ginkgo biloba, saw palmetto, panax, siberian ginseng, foeniculum vulgare, cimicifuga racemosa, licorice root, red clover, sage, sarsaparilla, sassafras, angelica sinensis achillea millefolium, anemone pratensis, angelica sinensis, glycyrrhiza glabra, hypericum perforatum, larrea, panax, piscidia erythrina, plantago psyllium, serenoa repens, symphytum, taraxacum officinale, trifolium pratense, turnera spp., tussilago farfara, valeriana officinalis, viburnum prunifolium, calendula officinalis;
(xxx) any one of the compounds exemplified in the present specification; and salts thereof.

According to further embodiments of the present invention, there is provided a method of treating, alleviating or preventing disorders of the skin, a body cavity or mucosal surface, where the disorder involves inflammation as one of its etiological factors. The method includes topically administering a foamed composition to a subject having the disorder, the foamed composition including a steroid, at least one organic carrier selected from a hydrophobic organic carrier, a polar solvent, an emollient and mixtures thereof, at a concentration of about 2% to about 50% by weight, about 0.1% to about 5% by weight of a surface-active agent, about 0.01% to about 5% by weight of a polymeric additive selected from a bioadhesive agent, a gelling agent, a film forming agent and a phase change agent and water, wherein the steroid is administered in a therapeutically effective amount.

Disorders suitable for treatment include vaginal disorders, vulvar disorders, anal disorders, disorders of a body cavity, ear disorders, disorders of the nose, disorders of the respiratory system, bacterial infections, fungal infections, viral infections, dermatosis, dermatitis, parasitic infections, disorders of hair follicles and sebaceous glands, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, chlamydia infection, gonorrhea infection, hepatitis B, herpes, HIV/AIDS, human papillomavirus (HPV), genital warts, bacterial vaginosis, candidiasis, cancroids, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, osteoarthritis, joint pain, hormonal disorder, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; sexual dysfunction in men and women, androgen deficiency; estrogen deficiency, growth disorders, hypogonadism, cancer, vasomotor symptoms, menopausal disorders, vulvar and vaginal atrophy, urethritis, hypoestrogenism, osteoarthritis, osteoporosis, uterine bleeding, hirsutism, virilization, ovarian tumors, hypothalamic pituitary unit diseases, testicular tumors, prostate cancer, hypopituitarism, Klinefelter's syndrome, testicular feminisation, orchitectomy, vasomotor symptoms (such as “hot flashes”) associated with the menopause, metabolic abnormalities and mood disturbances.

### BRIEF DESCRIPTION OF THE DRAWING

The invention is described with reference to the figure which is presented for the purpose of illustration and are not intended to be limiting of the invention.

FIG. 1 is a schematic illustration of an aerosol valve suitable for use in the aerosol packaging assembly according to in one or more embodiments of the invention.

FIG. 2A-2F are photomicrographs (×400, polarized light) of (A) the emulsion composition of Example 7 demonstrating that no crystals are observable; (B) the water phase of the composition of Example 7 following addition of a surfactant demonstrating that no crystals are observable; (C and D) the water phase of the composition of Example 7 prior to the addition of surfactant in which crystals are clearly visible; (E) betamethasone valerate powder used in the preparation of the composition of Example 7 and (F) a commercial betamethasone valerate 0.12% cream.

### DETAILED DESCRIPTION OF THE INVENTION

The present invention provides a therapeutic kit including a foamable steroid composition. The kit includes an aerosol packaging assembly having a container accommodating a pressurized product and an outlet capable of releasing the pressurized product as a foam.

Aerosol Packaging Assembly

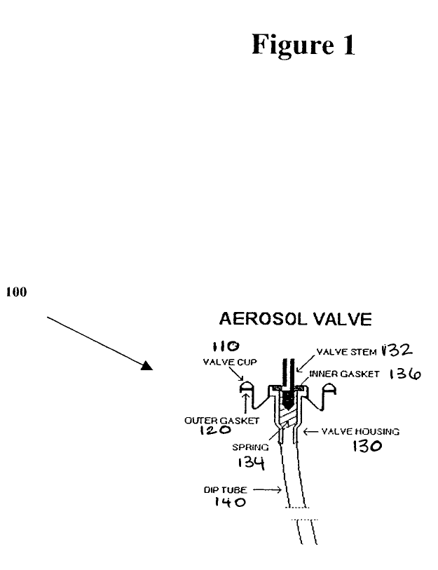

The aerosol packaging assembly typically includes a container suitable for accommodating a pressurized product and an outlet capable of releasing a foam. The outlet is typically a valve. FIG. 1 illustrates a typical aerosol valve 100. The valve is made up of the valve cup 110 typically constructed from tinplated steel, or aluminum, an outer gasket 120, which is the seal between the valve cup and the aerosol can (not shown), a valve housing 130, which contains the valve stem 132, spring 134 and inner gasket 136, and a dip tube 140, which allows the liquid to enter valve. The valve stem is the tap through which the product flows. The inner gasket 136 covers the aperture 150 (hole) in the valve stem. The valve spring 134 is usually made of stainless steel.

The valve stem is fitted with small apertures 150 (also termed “orifices” and “holes”), through which the product flows. Valves may contain one, two, three, four or more apertures, depending on the nature of the product to be dispensed. In the closed position, the aperture(s) is covered by the inner gasket. When the actuator is depressed it pushes the valve stem through the inner gasket, and the aperture(s) is uncovered, allowing liquid to pass through the valve and into the actuator.

The valve can have a stem with 1 to 4 apertures, or 1 to 2 apertures. Each aperture can have a diameter of about 0.2 mm to about 1 mm, or a diameter of about 0.3 mm to about 0.8 mm. The total aperture area, i.e., the sum of areas of all apertures in a given stem, is between about 0.01 mm² and 1 mm² or the total aperture area is between about 0.04 mm² and 0.5 mm².

In order to provide proper therapy, precise dosing is desired. According to one or more embodiments, the valve is attached, directly or through a tube, to a metered dose device for dispensing an accurate dose of drug in the form of a foam. The metered dose valve is selected to release a foam in a volume that provides an adequate therapeutic dose to the target site of the skin, a body surface, a body cavity or mucosal surface, e.g., the mucosa of the nose, mouth, eye, ear, respiratory system, vagina or rectum.

In one or more embodiments, the meter dose valve provides a unit dose of between about 10 μL and about 1000 μL of liquid. Assuming a representative foam density (specific gravity) of 0.06 g/mL, a 10 μL valve provides a foamed volume of about 0.17 mL, and a 1000 μL metered dose valve provides a foamed volume of about 17 mL. Thus, by selecting a specific metered dosing valve, adjusting the foam density by fine-tuning formulation parameters and adjusting the ratio between the liquid components of the composition and the propellant, one can design an adequate dosage form for a specific target site.

Pharmaceutical Composition

All % values are provided on a weight (w/w) basis.

According to one or more embodiments of the present invention, the foamable therapeutic composition for administration to the skin, a body surface, a body cavity or mucosal surface, e.g., the mucosa of the nose, mouth, eye, ear, respiratory system, vagina or rectum, e.g., the “target site” includes:
(1) a steroid, wherein the amount of the amount of the steroid is effective in the treatment of a disorder of the target site;
(2) at least one organic carrier selected from a hydrophobic organic carrier, a polar solvent, an emollient and mixtures thereof, at various concentrations, e.g., about 2% to about 5%; or about 5% to about 10%; or about 10% to about 20%; or about 20% to about 50% by weight;
(3) about 0.1% to about 5% by weight of a surface-active agent;
(4) about 0.01% to about 5% by weight of at least one polymeric agent selected from a bioadhesive agent, a gelling agent, a film forming agent and a phase change agent; and
(5) a liquefied or compressed gas propellant at a concentration of about 3% to about 25% by weight of the total composition.

Water and optional ingredients are added to complete the total mass to 100%. Upon release from an aerosol container, the foamable composition forms an expanded foam suitable for topical administration.

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butanol, iso-butanol, t-butanol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

In one or more embodiments, at least a portion of the steroid is suspended in the composition, yet, in other embodiments, the steroid is dissolved in the composition.

In one or more embodiments, the foam composition is formulated as an oil-in-water emulsion or oil-in-water microemulsion.

In one or more embodiments, the concentration of surface-active agent is from about 0.1% to about 5%, or from about 0.2% to about 2%.

In the context of the present invention, steroids are compounds possessing the skeleton of cyclopenta[a]phenanthrene or a skeleton derived therefrom by one or more bond scissions or ring expansions or contractions. Methyl groups are normally present at C-10 and C-13. An alkyl side chain may also be present at C-17. Sterols are steroids carrying a hydroxyl group at C-3 and most of the skeleton of cholestane. Additional carbon atoms may be present in the side chain.

Steroids are numbered and rings are lettered as in formula 1. If one of the two methyl groups attached to C-25 is substituted it is assigned the lower number (26); if both are substituted, that carrying the substituent cited first in the alphabetical order is assigned the lower number.

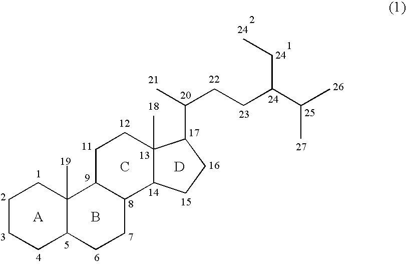

The steroids can have substituents on the steroid side chain as exemplified in formula 4-7:

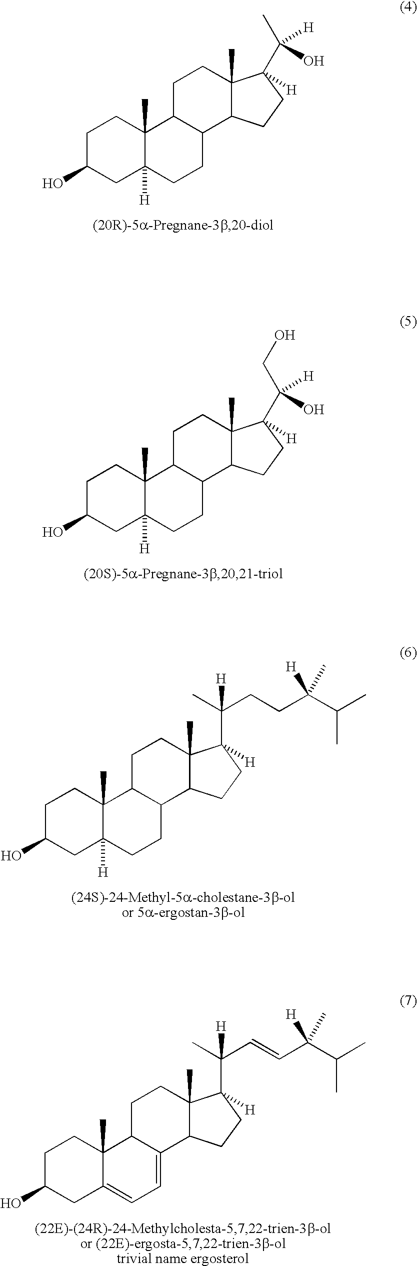

The steroids can have the formalae as exemplified in formula 9-18. In one or more embodiments, the steroid or sterol has unsaturation or substitution at C-17, as exemplified by gonane, e.g., formulae 9 and 10, estrange (also termed oestrane), e.g. formulae 11 and 12, and androstane, e.g., formulae 13 and 14. In one or more embodiments, the steroid or sterol has methyl groups at both C-10 and C-13 and a side chain R at C-17 (formulae 15 and 16), as exemplified in Table 1.

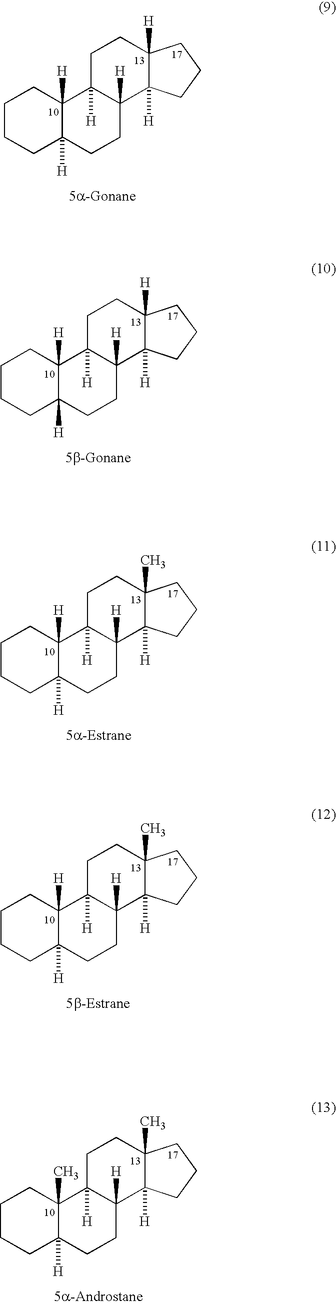

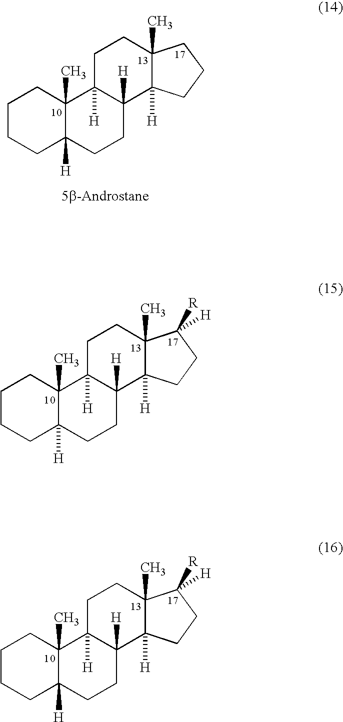

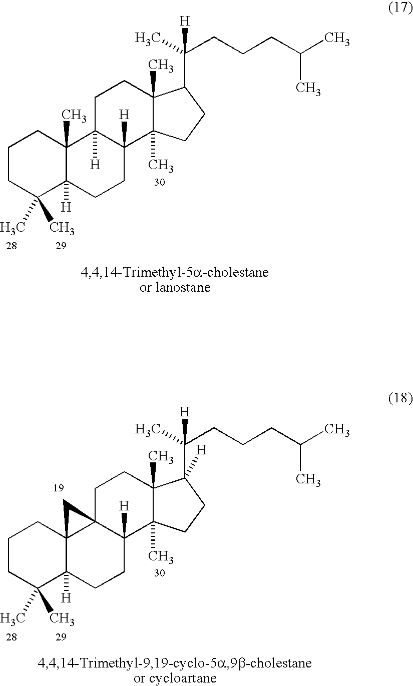

TABLE 1
Hydrocarbons with side chain at C-17

| Side chain | 5^{α}-Series (15) | 5^{β}-Series (16) |
|---|---|---|
| 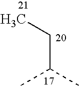 | 5^{α}-pregnane (allopregnane) | 5^{β}-pregnane |
| 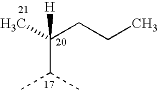 | 5^{α}-cholane (allocholane) | 5^{β}-cholane |
| 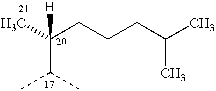 | 5^{α}-cholestane | 5^{β}-cholestane (coprostane) |
| 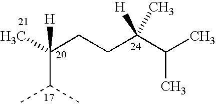 | 5^{α}-ergostane | 5^{β}-ergostane |
| 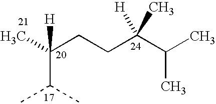 | 5^{α}-campestane | 5^{β}-campestane |
| 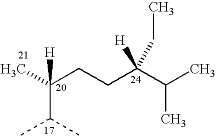 | 5^{α}-poriferastane | 5^{β}-poriferastane |
| 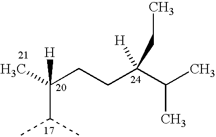 | 5^{α}-stigmastane | 5^{β}-stigmastane |
| 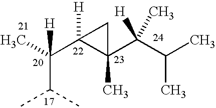 | 5^{α}-gorgostane | 5^{β}-gorgostane |

Examples of unsaturated steroids and sterols are provided in formulae 19-22:

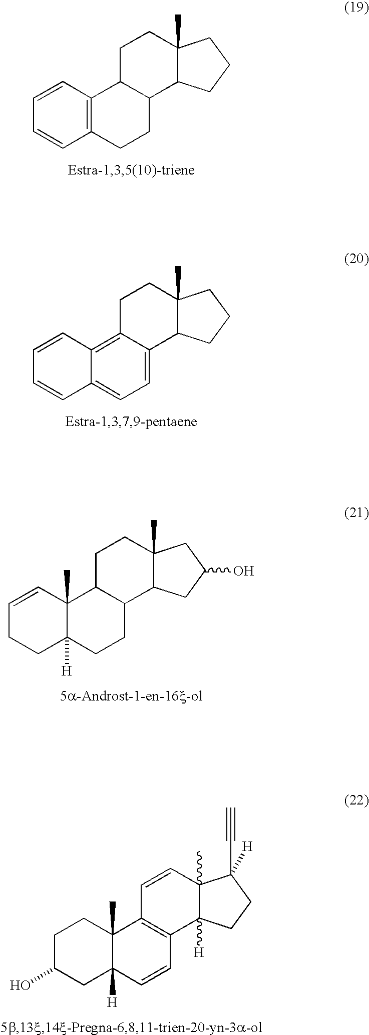

The stereochemistry of double bonds in the side chain is indicated using the E,Z convention. The same applies to the seco compounds of the vitamin D series (example in formula 23). In certain cases, the steroid has two carbon chains attached at position 17, e.g. 17-methyl-5α-pregnane 24,17-methyl-5α,17β-pregnane 25,17-ethyl-5-cholestane, 17-ethyl-5-pregnane and 17-ethyl-5-cholestane and 17-(2-bromoethyl)-5α,17α-cholestane 26. Other examples of a steroid that has two carbon chains attached at position 17, are 17,17-dimethyl-5α-androstane 27 and 17β-methyl-17α-propyl-5α-androstane 28. In certain embodiments, the carbon skeleton of a steroid a carbon atom is replaced by a hetero atom, as exemplified by 17β-hydroxy-4-oxaandrost-5-en-3-one 29. Yet, in additional embodiments, an additional ring is formed by means of a direct link between any two carbon atoms of the steroid ring system or the attached side chain, as exemplified by formulae 30, 31 and 32.

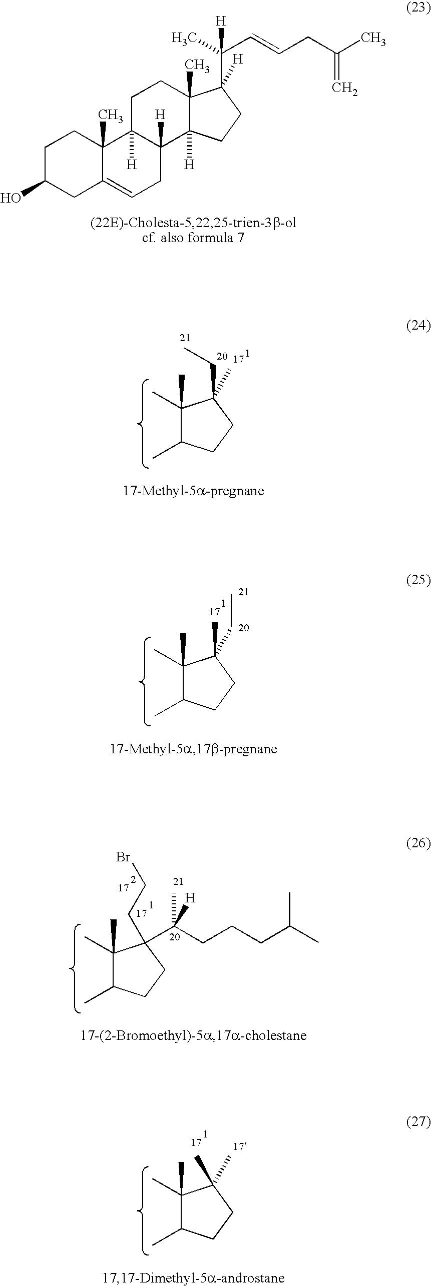

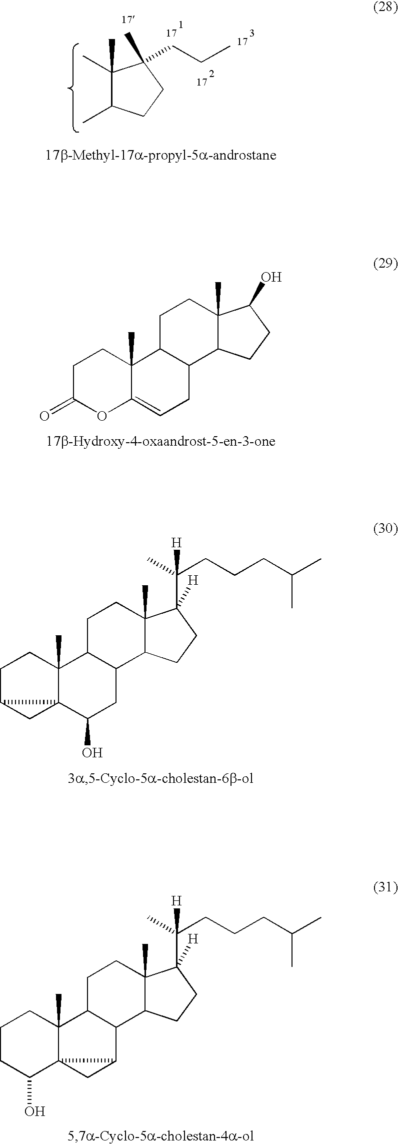

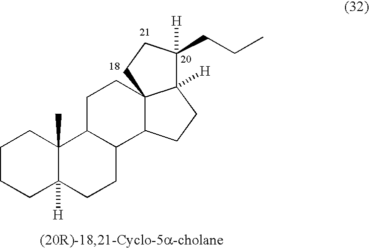

Many important naturally occurring steroids contain one or more additional heterocyclic ring(s), fused or attached to ring D, formed by modifications of the side chain. These steroids can be grouped into the following families: (a) cardanolides, e.g., 5β-cardanolide 33, 3β,14-dihydroxy-5β-card-20(22)-enolide (digitoxigenin) 34 and 3β,5,14-trihydroxy-19-oxo-5β-card-20(22)-enolide (strophanthidin) 35, as well as epoxycardanolides, containing a 14,21- or a 16,21-oxygen bridge, as shown in 36, (b) bufanolides, e.g., structures 37-39, (c) spirostans, e.g., structures 40-43, (d) furostans, e.g., structures 44-45, and (e) steroid alkaloids.

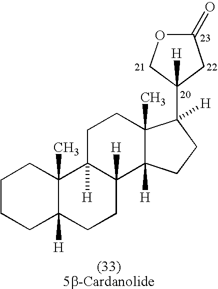

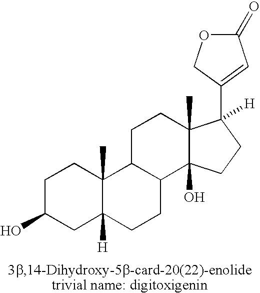

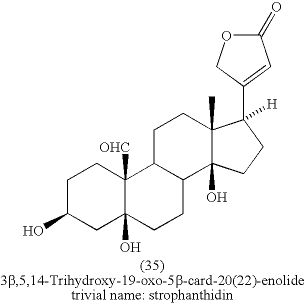

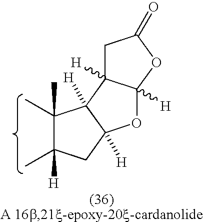

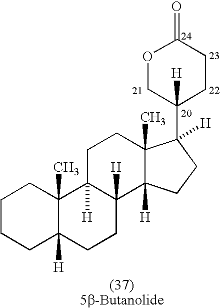

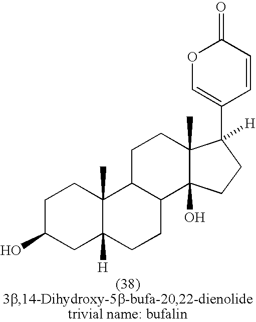

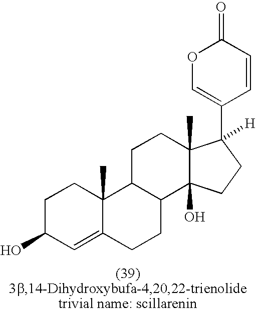

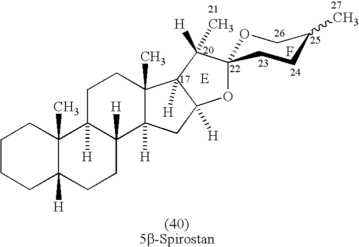

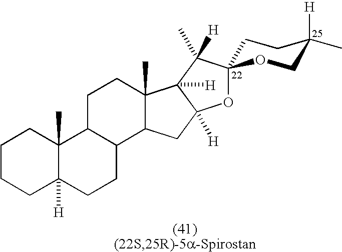

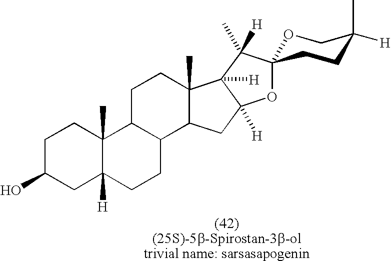

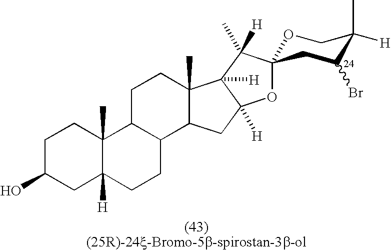

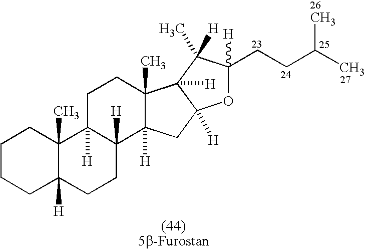

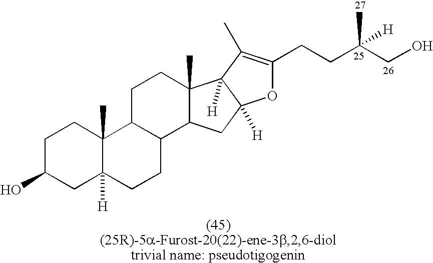

Several biologically important steroids are derivatives of the parent hydrocarbons carrying various functional groups. Some of the common functional groups include but are not limited to halogens, alkyl groups, aryl groups, benzyl groups, carboxy groups and alkoxy groups.

In one or more embodiments, the steroid is selected from the group consisting of an acid, a salt of an acid, as exemplified in formulae 46-49, and esters, as exemplified in formulae 50 and 51. In one or more embodiments, the steroid is a lactone, as exemplified in formulae 52-54.

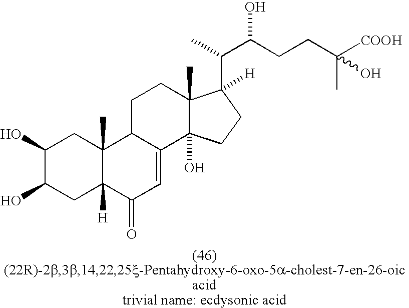

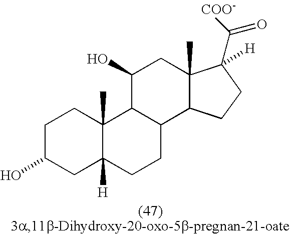

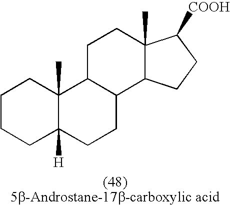

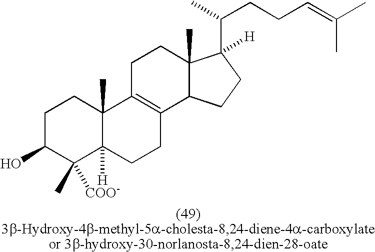

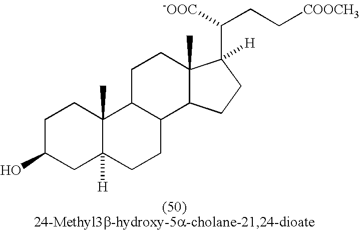

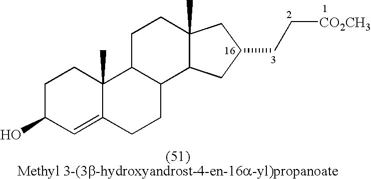

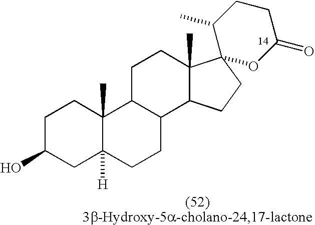

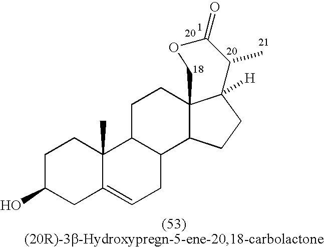

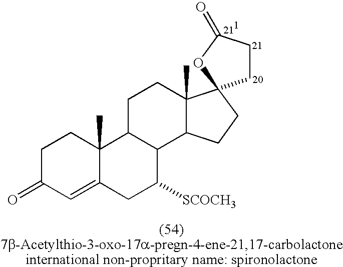

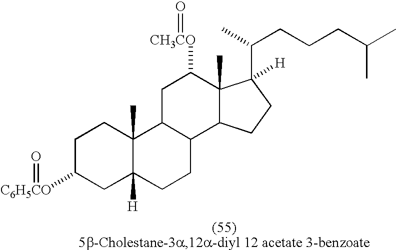

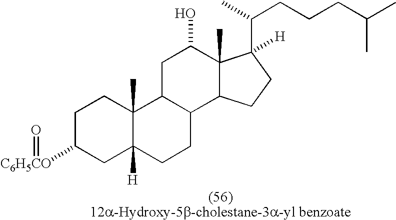

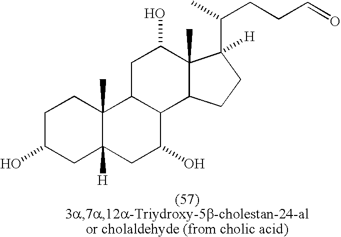

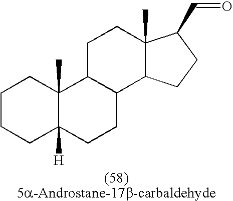

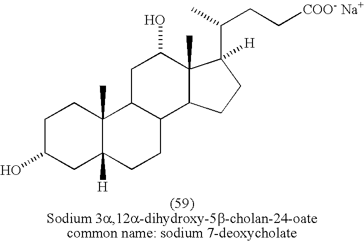

In one or more embodiments, the steroid is an ester of a steroid alcohol, as exemplified by 5-cholestan-3-yl acetate, 5-cholestane-3,12-diyl diacetate, 3-oxoandrost-4-en-17-yl acetate (trivial name testosterone acetate), 17-hydroxy-20-oxopregn-5-en-3-yl sulfate, 3-acetoxy-5-cardanolide, 3-benzoyloxy-11-hydroxy-20-oxo-5-pregnan-21-oate (monobenzoate of 47), 3-acetoxy-5-cholano-24,17-lactone (acetate of 52), 3-O-acetylcholic acid, 17-O-benzoylestradiol-17, 3-O-linolenoylcholesterol, as well as in formulae 55 and 56.

In one or more embodiments, the steroid is an oxo compound. The oxo compound can be an aldehde, as exemplified by 5-androstan-19-al, 5-cholan-24-al, 3-formyl-5-cholan-24-oic acid and by formulae 57 and 58, or a ketone, as exemplified by 5-androstan-3-one, pregn-5-ene-3,20-dione and 11-oxo-5-cholan-24-oic acid.

In one or more embodiment, the steroid is an alcohol as exemplified by 5-cholestane-3,11-diol, 3-hydroxy-5-androstan-17-one (trivial name: androsterone) and by formulae 59.

In additional embodiments, the steroid is an amine as exemplified by androst-5-en-3-amine and formula 60, an ether as exemplified by 17-methoxyandrost-4-en-3-one, (20S)-3,17,20-trimethoxy-5-pregnane, (20S)-3,17-dimethoxy-5-pregnan-20-ol, 21-O-methylcortisol and formula 61, an acetal or a ketal of an oxo steroid (also named as dialkoxy steroids) as exemplified by 3,3-dimethoxycholest-4-ene, 2,3-(methylenedioxy)pregn-5-ene and formula 62.

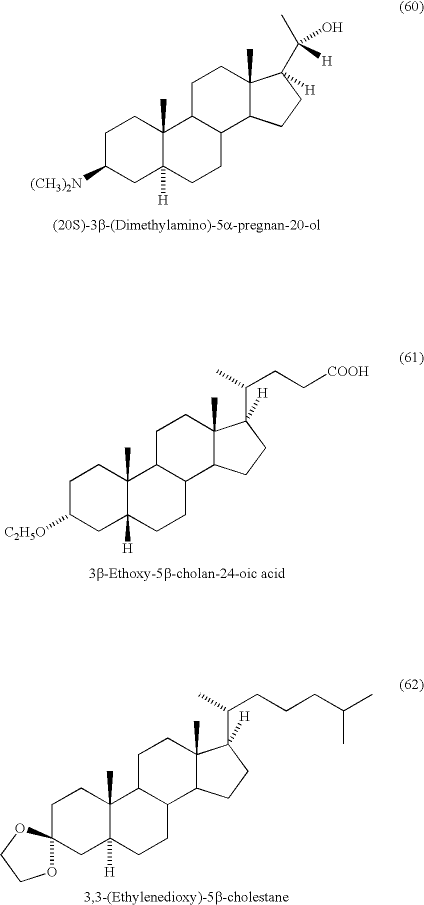

Examples of trivial names retained for important steroid derivatives, these being mostly natural compounds of significant biological activity, are given in Table 2.

TABLE 2
Trivial names of some important steroid derivatives

| Trivial name | Systematic steroid name |
|---|---|
| Aldosterone | 18,11-hemiacetal of 11^{β},21-dihydroxy-3,20- |
| | dioxopregn-4-en-18-al or 11^{β},18-epoxy-18^{ξ},21- |
| | dihydroxypregn-4-ene-3,20-dione |
| Androsterone | 3α-hydroxy-5α-androstan-17-one |
| Brassinolide | (22R,23R)-2α,3α,22,23-tetrahydroxy-6,7-seco-5α- |
| | cmpestano-6,7-lactone |
| Calcidiol (93) | (5Z,7E)-(3S)-9,10-secocholesta-5,7,10(19)-triene-3, |
| | 25-diol |
| Calciol = | (5Z,7E)-(3S)-9,10-secocholesta-5,7,10(19)-trien-3-ol |
| cholecalciferol (92) | |
| Calcitriol (94) | (5Z,7E)-(1S,3R)-9,10-secocholesta-5,7,10(19)- |
| | triene-1,3,25-triol |
| Cholesterol | cholest-5-en-3^{β}-ol |
| Cholic acid | 3α,7α,12α-trihydroxy-5^{β}-cholan-24-oic acid |
| Corticosterone | 11^{β},21-dihydroxypregn-4-ene-3,20-dione |
| Cortisol | 11^{β},17,21-trihydroxypregn-4-ene-3,20-dione |
| Cortisol acetate | 21-O-acetylcortisol |
| Cortisone | 17,21-dihydroxypregn-4-ene-3,11,20-trione |
| Cortisone acetate | 21-O-acetylcortisone |
| Deoxycorticosterone | 21-hydroxypregn-4-ene-3,20-dione (i.e. the 11- |
| | deoxy derivative of corticosterone) |
| Ecdysone | (22R)-2^{β},3^{β},14α,22,25-pentahydroxy-5^{β}- |
| | cholest-7-en-6-one |
| Ercalciol = | (5Z,7E,22E)-(3S)-9,10-secoergosta-5,7,10(19),22- |
| ergocalciferol | tetren-3-ol |
| Ergosterol (7) | (22E)-ergosta-5,7,22-trien-3^{β}-ol |
| Estradiol-17α | estra-1,3,5(10)-triene-3,17α-diol |
| Estradiol-17^{β} | estra-1,3,5(10)-triene-3,17^{β}-diol |
| Estriol | estra-1,3,5(10)-triene-3,16α,17^{β}-triol |
| Estrone | 3-hydroxyestra-1,3,5(10)-trien-17-one |
| Lanosterol | lanosta-8,24-dien-3^{β}-ol |
| Lithocholic acid | 3α-hydroxy-5^{β}-cholan-24-oic acid |
| Progesterone | pregn-4-ene-3,20-dione |
| Pseudotigogenin | (25R)-5α-furost-20(22)-ene-3^{β},26-diol |
| Sarsasapogenin | (25S)-5^{β}-spirostan-3^{β}-ol |
| Smilagenin | (25R)-5^{β}-spirostan-3^{β}-ol |
| Testosterone (63) | 17^{β}-hydroxyandrost-4-en-3-one |
| Tigogenin | (25R)-5α-spirostan-3^{β}-ol |

Additional non-limiting examples of steroids that are applicable according to the present invention are provided in formulae 63-79.

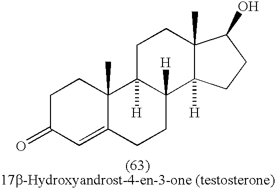

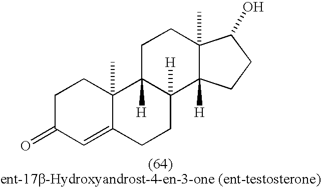

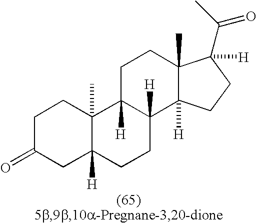

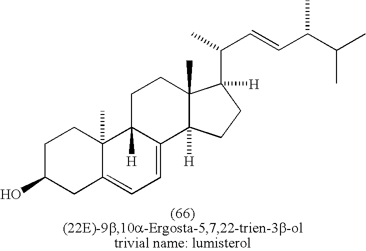

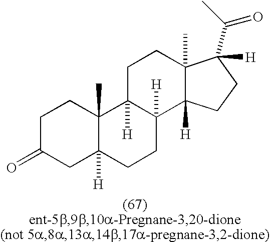

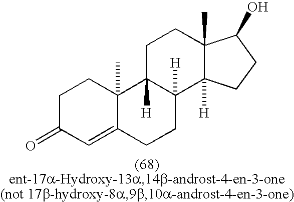

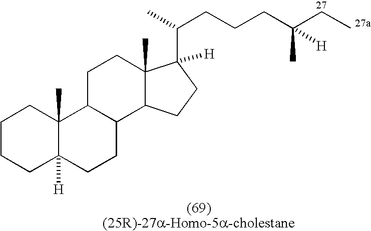

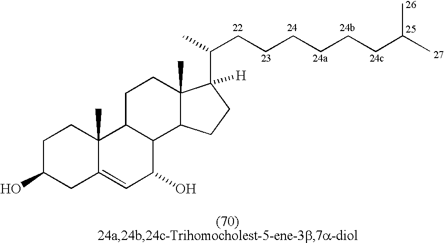

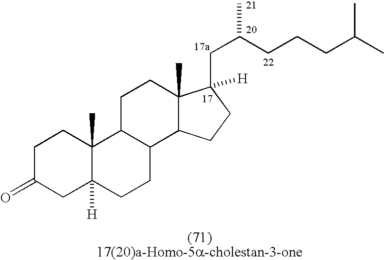

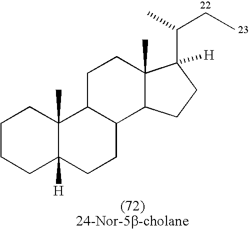

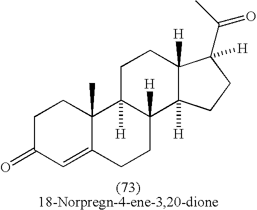

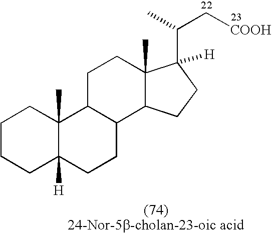

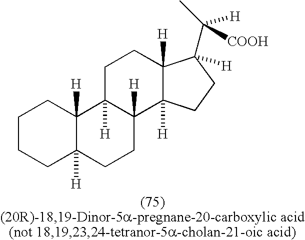

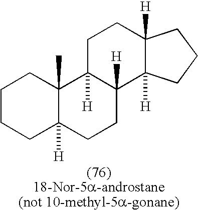

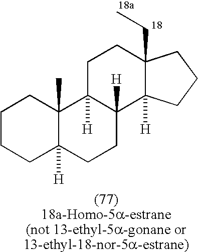

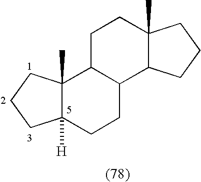

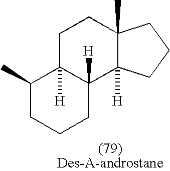

In one or more embodiments according to the present invention, the steroid is a compound, in which one or more of the cyclopenta[a]phenanthrene rings is contracted by loss of an unsubstituted methylene group, as exemplified by 4-nor-5-androstane (78), where C-4 is missing. In other embodiments one or more of the cyclopenta[a]phenanthrene rings is expanded by inclusion of a methylene group, as exemplified by formulae 80-86.

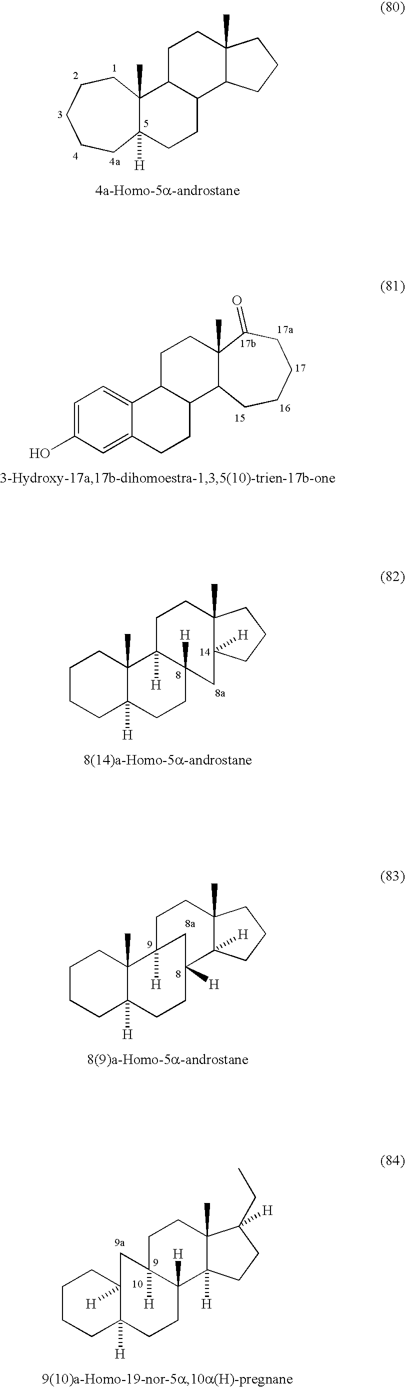

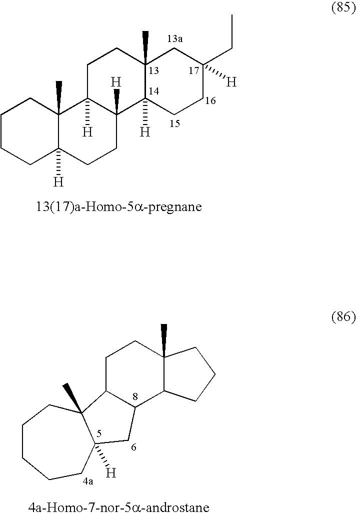

In one or more embodiments, the steroid contains additional rings that are formed within, or on, a steroid nucleus. In additional embodiments, the steroids contains a bivalent bridge such as —O—O—, —[CH₂]ₙ—, linking non-adjacent ring positions as exemplified by formulae 99-102.

In one or more embodiments, the steroid contains a cyclopenta[a]phenanthrene skeleton and a carbocyclic or heterocyclic ring component fused to it, as exemplified by formulae 103-111, and in other embodiments, an additional ring is linked to the cyclopenta[a]phenanthrene skeleton through a spiro system, as exemplified by formula 112.

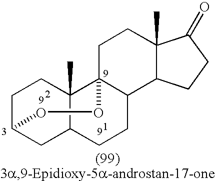

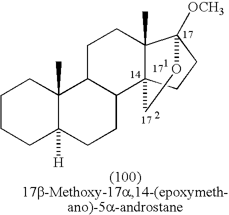

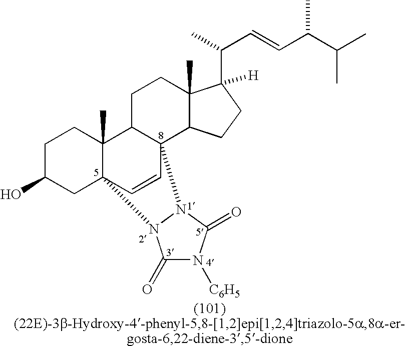

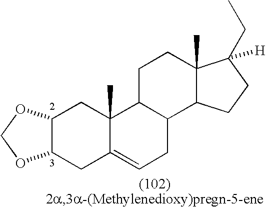

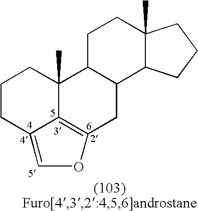

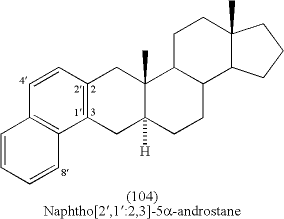

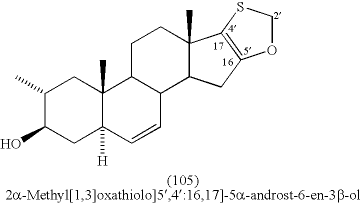

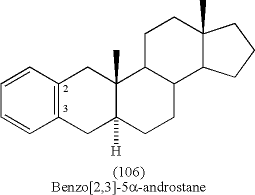

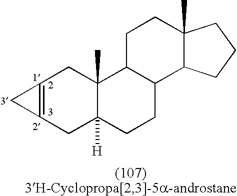

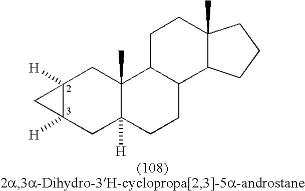

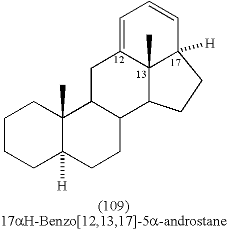

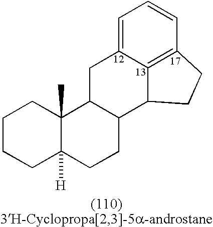

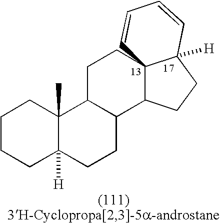

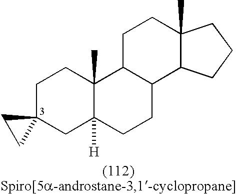

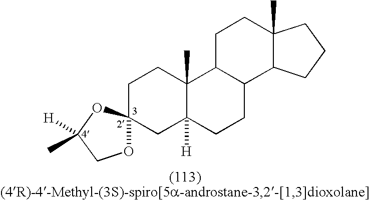

Yet, in certain embodiments, two or more steroid molecules are linked together covalently, as exemplified by formulae 3a and 3b.

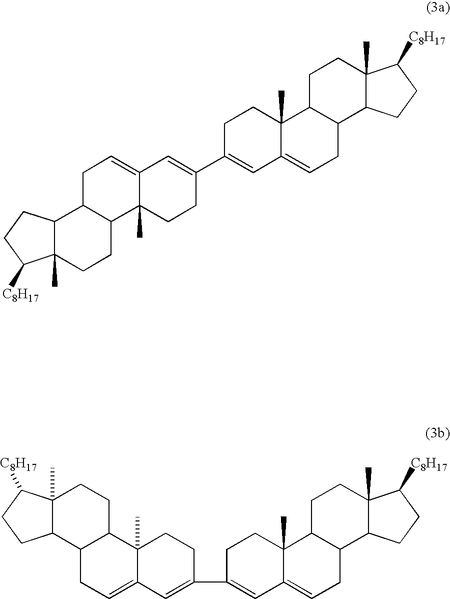

Table 3 provides examples of steroids that are useful according to the present invention.

TABLE 3
Exemplary steroids that are useful according to the present invention.

| | | Molecular |
|---|---|---|
| Trivial name | Chemical name | formula |
| Acrihellin | 5,14-dihydroxy-3^{β}-[(3-methylcrotonoyl)oxy]-19-oxo- | C₂₉H₃₈O₇ |
| | 5^{β}-bufa-20,22-dienolide | |
| Actodigin | 3^{β}-(^{β}-D-glucopyranosyloxy)-14-hydroxy-24-nor-5^{β}, | C₂₉H₄₄O₉ |
| | 14^{β}-chol-20(2)-eno-21,23-lactone | |
| Alfacalcidol | (5Z,7E)-(1S,3R)-9,10-secocholesta-5,7,10(19)- | C₂₇H₄₄O₂ |
| | triene-,3-diol | |
| Betamethasone | 9-fluoro-11^{β},17,21-trihydroxy-16^{β}-methylpregna- | C₂₂H₂₉FO₅ |
| | 1,4-diene-3,20-dione | |
| Canrenone | 3-oxo-17α-pregna-4,6-diene-21,17-carbolactone | C₂₂H₂₈O₃ |
| Clomegestone | 6-chloro-17-hydroxy-16α-methylpregna-4,6-diene- | C₂₂H₂₉ClO₃ |
| | 3,20-dione | |
| Cyproterone | 6-chloro-1^{β},2^{β}-dihydro-17-hydroxy-3′H- | C₂₂H₂₇ClO₃ |
| | cyclopropa[1,2]pregna-4,6diene-3,20-dione | |
| Dexamethasone | 9-fluoro-11^{β},17,21-trihydroxy-16α-methylpregna- | C₂₂H₂₉FO₅ |
| | 1,4-diene-3,20-dione | |
| Disogluside | (25R)-3^{β}-(^{β}-D-glucopyranosyloxy)spirost-5-ene | C₃₃H₅₂O₈ |
| Ethinylestradiol | 19-nor-17α-pregna-1,3,5(10)-trien-20-yne-3,17-diol | C₂₀H₂₄O₂ |
| Fluazacort | 21-acetoxy-9-fluoro-11^{β}-hydroxy-2′-methyl-16bH- | C₂₅H₃₀FNO₆ |
| | oxazolo[5′,4′:16,17]pegna--1,4-diene-3,20-dione | |
| Fluocortin | 6α-fluoro-11^{β}-hydroxy-16α-methyl-3,20- | C₂₂H₂₇FO₅ |
| | dioxopregna-1,4-dien-21-oic acid | |
| Fusidic Acid | (17Z)-ent-16α-acetoxy-3^{β},11^{β}-dihydroxy-4^{β},8,14- | C₃₁H₄₈O₆ |
| | trimethyl-18-nor-5^{β},10α-cholesta--17(20),24-dien- | |
| | 21-oic acid | |
| Gestrinone | 17-hydroxy-18α-homo-19-nor-17α-pregna-4,9,11- | C₂₁H₂₄O₂ |
| | trien-20-yn-3-one | |
| Halometasone | | |
| | 2-chloro-6α,9-difluoro-11^{β},17,21-trihydroxy-16α- | C₂₂H₂₇ClF₂O₅ |
| | methylpregna-1,4-diene-3,20-dione | |
| Hydrocortisone | 11^{β},17,21-trihydroxypregn-4-ene-3,20-dione | C₂₁H₃₀O₅ |
| Mebolazine | 17^{β}-hydroxy-2α,17-dimethyl-5α-androstan-3-one | C₄₂H₆₈N₂O₂ |
| | azine | |
| Medroxyprogesterone | 17-hydroxy-6α-methylpregn-4-ene-3,20-dione | C₂₂H₃₂O₃ |
| Meproscillarin | 3^{β}-(6-deoxy-4-O-methyl-α-L-mannopyranosyloxy)- | C₃₁H₄₄O₈ |
| | 14-hydroxybufa-4,20,22-rienolide | |
| Mespirenone | 7α-acetylthio-15α,16α-dihydro-3-oxo-3′H- | C₂₅H₃₀O₄S |
| | cyclopropa[15,1]-17α-pregna--1,4-diene-21,17- | |
| | carbolactone | |
| Mestranol | 3-methoxy-19-nor-17α-pregna-1,3,5(10)-trien-20- | C₂₁H₂₆O₂ |
| | yn-17-ol | |
| Naflocort | 9-fluoro-1′,4′-dihydro-11^{β},21-dihydroxy-16bH- | C₂₉H₃₃FO₄ |
| | naphtho[2′,3′:16,17]prena--1,4-diene-3,20-dione | |
| Norenthisterone | 17-hydroxy-19-nor-17α-pregn-4-en-20-yn-3-one | C₂₀H₂₆O₂ |
| Norgesterone | 17-hydroxy-19-nor-17α-pregna-5(10),20-dien-3-one | C₂₀H₂₈O₂ |
| Norgestrel | rac-17-hydroxy-18α-homo-19-nor-17α-pregn-4-en- | C₂₁H₂₈O₂ |
| | 20-yn-3-one | |
| Oxandrolone | 17^{β}-hydroxy-17α-methyl-2-oxa-5α-androstan-3- | C₁₉H₃₀O₃ |
| | one | |
| Oxymetholone | 17^{β}-hydroxy-2-(hydroxymethylene)-17α-methyl-5α- | C₁₉H₂₈O₃ |
| | androstan-3-one | |
| Pancuronium | 1,1′-(3α,17^{β}-diacetoxy-5α-androstane-2^{β},16^{β}- | C₃₅H₆₀Br₂N₂O₄ |
| bromide | diyl)bis(-methylpiperidinium) dibromide | |
| Prednisolone | 11^{β},17,21-trihydroxypregna-1,4-diene-3,20-dione | C₂₁H₂₈O₅ |
| Prednisone | 17,21-dihydroxypregna-1,4-diene-3,11,20-trione | C₂₁H₂₆O₅ |
| Proscillardin | 3^{β}-(6-deoxy-α-L-mannopyranosyloxy)-14- | C₃₀H₄₂O₈ |
| | hydroxybufa-4,20,22-trienolide | |
| Roxibolone | 11^{β},17^{β}-dihydroxy-17α-methyl-3-oxoandrosta-1,4- | C₂₁H₂₈O₅ |
| | diene-2-carboxylic acid | |
| Spironolactone | 7α-acetylthio-3-oxo-17α-pregn-4-ene-21,17- | C₂₄H₃₂O₄S |
| | carbolactone | |
| Timobesone | S-methyl 9-fluoro-11^{β},17α-dihydroxy-16^{β}-methyl-3- | C₂₂H₂₉FO₄S |
| | oxoandrosta--1,4-diene-17^{β}-carbothioate | |
| Triamcinolone | 9-fluoro-11^{β},16α,17,21-tetrahydroxypregna-1,4- | C₂₁H₂₇FO₆ |
| | diene-3,20-dione | |
| Ursodeoxycholic | 3α,7^{β}-dihydroxy-5^{β}-cholan-24-oic acid | C₂₄H₄₀O₄ |
| acid | | |

Mixtures of these steroids may also be employed according to the present invention.

Solubility of the steroid is an important factor in the development of a stable foamable composition according to the present invention.

For definition purposes, in the context of the present invention, the descriptive terminology for solubility according to the US Pharmacopoeia (USP 23, 1995, p. 10), the European Pharmacopoeia (EP, 5^{th} Edition (2004), page 7) and several other textbooks used in the art of pharmaceutical sciences (see for example, Martindale, The Extra Pharmacopoeia, 30^{th} Edition (1993), page xiv of the Preface; and Remington's Pharmaceutical Sciences, 18^{th} Edition (1990), page 208) is adapted:

| | Parts of Solvent Required |
|---|---|
| Descriptive Term | for 1 Part of Solute |
| Very soluble | Less than 1 |
| Freely soluble | From 1 to 10 |
| Soluble | From 10 to 30 |
| Sparingly soluble | From 30 to 100 |
| Slightly soluble | From 100 to 1,000 |
| Very slightly soluble | From 1,000 to 10,000 |
| Practically insoluble or Insoluble | 10,000 and over |

Thus, in one or more embodiments, the steroid is “soluble”, “freely soluble” or “very soluble” (as defined above) in the aqueous phase of the emulsion. In other embodiments, the agent possesses hydrophobic characteristics and the steroid is “soluble”, “freely soluble” or “very soluble” in the oil phase of the emulsion. Yet, in certain cases, the steroid is “very slightly soluble”, “slightly soluble” or “sparingly soluble” in either the water phase or oil phase of the emulsion.

Yet, in one or more embodiments, the steroid is insoluble i.e., “requires 10,000 parts or more of a solvent to be solubilized”, in either the water phase of the composition, or the oil phase of the composition, but not in both.

It has been surprisingly discovered that while insoluble in water, a steroid can be solubilized in the aqueous phase of the emulsion (prior to combining the oil and aqueous phases to form an emulsion), by adding a surfactant and optionally, a polymeric agent, without the need of an “organic co-solvent”. It has been further surprisingly discovered that including a steroid in a foamable emulsion that is insoluble both in water and in the oil phase of the composition can result in a composition in which the steroid is dissolved, with no relation to the addition of an “organic co-solvent”. Thus, in further embodiments, the steroid is solubilized in the emulsion, although it is insoluble both in water and in the oil phase of the composition. In more specific embodiments, the composition of the present invention contains a solubilized steroid, although the composition does not contain an “organic co-solvent”. An “organic co-solvent”, is one of the group consisting of an ester of a fatty acid for example a C12-C15 alkyl benzoate, a medium to long chain alcohol, an aromatic and/or alkyl pyrollidinone, an aromatic and/or alkyl, and/or cyclic ketone, an aromatic and/or alkyl, and/or cyclic ether, substituted and/or unsubstituted single or multiple ring aromatic, straight chain and/or branched chain and/or cyclic alkane or silicone.

In yet additional embodiments, the steroid is not fully dissolved in either the aqueous phase or the oil phase of the emulsion concurrently, and thus, it is suspended in the emulsion, i.e., at least a portion of the steroid portion remains in solid state in the final composition. In such a case, the polymeric agents that are listed herein serve as suspension-stabilizing agents to stabilize the composition.

In certain embodiments of the present invention, the composition and properties of the aqueous phase of the emulsion (e.g., pH, electrolyte concentration and chelating agents) and/or the composition of the oil phase of the emulsion are adjusted to attain a desirable solubility profile of the active agent.

The steroid is included in the composition of the present invention in a concentration that provides a desirable ratio between the efficacy and safety. Typically, steroids are included in the composition in a concentration between about 0.005% and about 12%. However, in some embodiments, the concentration is between about 0.005% and about 0.5%, in other embodiment between about 0.5% and about 2%, and in additional embodiments between about 2% and about 5% or between about 5% and about 12%.

In one or more embodiments, the steroid possesses immunomodulating and/or anti-inflammatory properties. Without being bound to a specific theory, immunomodulating and/or anti-inflammatory steroids act, among other mechanisms, through inhibition of the activity of phospholipase A₂. They also may have anti-proliferative effects on keratinocytes and other cell types. They can suppress collagen synthesis by fibroblasts, but this may lead to adverse effects. Anti-inflammatory steroids are roughly grouped according to relative anti-inflammatory activity, but activity may vary considerably depending upon the vehicle, the site of application, disease, the individual patient and whether or not an occlusive dressing is used, as exemplified in Table 4.

TABLE 4
Exemplary anti-inflammatory steroids that are useful
according to the present invention.

| | | Typical concentration |
|---|---|---|
| Relative Potency | Generic Name | in topical products |
| Low Potency | Hydrocortisone | 0.5%-1% |
| | hydrocortisone acetate | 0.5-1.0% |
| | Desonide | 0.02-0.2% |
| Medium Potency | Betamethasone valerate | 0.05%-0.1% |
| | Prednicarbate | 0.02-0.2% |
| | Clobetasone-17-butyrate | 0.05% |
| | Flucinonide | 0.01%-0.05% |
| | Fluocinolone acetonide | 0.01-0.01% |
| | Alcometasone dipropionate | 0.01% |
| | Mometasone furoate | 0.1% |
| | Triamcinolone acetonide | 0.025%-0.1% |
| High Potency | Betamethasone-17-benzoate | 0.025% |
| | Methylprednisolone aceponate | 0.1% |
| | Betamethasone dipropionate | 0.025%, 0.05% |
| | Halcinonide | 0.1% |
| | Triamcinolone acetonide | 0.5% |
| Highest Potency | Halobetasol | 0.05% |
| | Clobetasol-17-propionate | 0.05% |

In one or more embodiments, the steroid is selected from the group of low-potency anti-inflammatory steroids, medium potency anti-inflammatory steroids and high potency anti-inflammatory steroids.

In one or more embodiments, the anti-inflammatory steroid is included in the composition at a concentration between about 0.005% and about 1%.

The McKenzie vasoconstrictor assay, as described, for example, in the British Journal of Dermatology 1975; 93:563-71 and versions thereof, has been the primary method used for classifying the potency of a product, containing an anti-inflammatory steroids. Thus, in one or more embodiments, the anti-inflammatory steroid is a steroid that positively affects the vasoconstrictor assay.

In one or more embodiments, the steroid is a hormone. Hormones are known to affect a variety of biological processes in any organism, and thus, their inclusion in the composition of the present invention, which is intended for local treatment of the skin, the vagina, the rectum as well as other body surfaces and cavities provided an advantageous treatment modality. Such compositions containing hormones can be further administered systemically, via the transdermal or transmucosal route, in order to alleviate a disorder that is affected by the specific hormone, or in order to tune the hormonal balance of the body in order to attain certain effects controlled by hormones, such as contraception and birth induction.

In one or more embodiments, the steroid hormone is a male hormone or an androgen. Non-limiting examples of male hormones/androgens include testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone (also termed prasterone and DHEA), androstenedione, androstanediol, androsterone, androstenolone, prasterone enantate, prasterone sodium sulfate, ormeloxifene, mesterolone, fluoxymesterone, methyltestosterone, gestrinone, delmadinone, delmadinone acetate, chlormadinone, chlormadinone acetate, danazol and testolactone.

In one or more embodiments, the steroid hormone is a female hormone or an estrogen. Non-limiting examples of female hormones/estrogens include estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate and equilin.

In one or more embodiments, the steroid hormone is a progestogen. Non-limiting examples of progestogens include progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlormadinone acetate, dienogest, drospirenone, lynestrenol, tybolone, cyproterone acetate, megestrol acetate, nomegestrol acetate.

Yet, in additional embodiments, the steroid an inhibitor of a steroid hormone. Non-limiting examples of such inhibitors are finasteride, dutasteride and spironolactone.

In one or more embodiments, the steroid is a vitamin D. The term vitamin D is used to describe all steroids that exhibit qualitatively the biological activity of calciol (vitamin D₃). Non limiting examples of vitamin D compounds are provided in Table 5.

TABLE 5
Examples of vitamin D compounds

| Vitamin D name | Systematic steroid name |
|---|---|
| Cholecalciferol (also termed calciol, | (5Z,7E)-(3S)-9,10-seco-5,7,10(19)- |
| cholecalciferol, vitamin D₃ and colecalciferol) | cholestatrien-3-ol |
| 25-Hydroxycholecalciferol (also termed | (5Z,7E)-(3S)-9,10-seco-5,7,10(19)- |
| calcidiol | cholestatriene-3,25-diol |
| 1α,25-Dihydroxycholecalciferol (also termed | (5Z,7E)-(1S,3R)-9,10-seco-5,7,10(19)- |
| calcitriol) | cholestatriene-1,3,25-triol |
| Ergocalciferol (also termed ercalciol and | (5Z,7E,22E)-(3S)-9,10-seco-5,7,10(19),22- |
| ergocalciferol) | ergostatetraen-3-ol |
| 1α,25-Dihydroxyergocalciferol (also termed | (5Z,7E,22E)-(1S,3R)-9,10-seco- |
| ercalcitriol) | 5,7,10(19),22-ergostatetraen-1,3,25-triol |
| 22,23-Dihydroergocalciferol (also termed | (5Z,7E)-(3S)-9,10-seco-5,7,10(19)- |
| (24S)-methylcalciol and 22,23- | ergostatrien-3-ol |
| dihydroercalciol) | |
| 1α,24R,25-Trihydroxycholecalciferol (also | (5Z,7E)-(1S,3R,24R)-9,10-seco-5,7,10(19)- |
| termed calcitetrol) | cholestatriene-1,3,24,25-tetrol |
| Previtamin D₃ (also termed precalciferol and | (6Z)-(3S)-9,10-seco-5(10),6,8-cholestatrien- |
| (6Z)-tacalciol) | 3-ol |
| Tachysterol₃ (also termed tacalciol) | (6E)-(3S)-9,10-seco-5(10),6,8-cholestatrien- |
| | 3-ol |
| Isovitamin D₃ (also termed (5E)-isocalciol) | (5E,7E)-(3S)-9,10-seco-1(10),5,7- |
| | cholestatrien-3-ol |
| Dihydrotachysterol₃ (also termed | (5E,7E)-(3S,10S)-9,10-seco-5,7- |
| dihydroercalciol) | cholestadien-3-ol |

Further examples of vitamin D compounds include, but are not limited to (1S)-Hydroxycalciol (also termed 1α-hydroxycholecalciferol and alfacaleidol), (24R)-Hydroxycalcidiol (also termed 24(R),25-dihydroxycholecalciferol), 25-Fluorocalciol (also termed 25-fluorocholecalciferol), Ercalcidiol (also termed 25-hydroxyergocalciferol), Ertacalciol (also termed tachysterol₂, (5E)-Isocalciol (also termed isovitamin D₃, 22,23-Dihydroercalciol), (24S)-methylcalciol (also termed vitamin D₄), (5E)-(10S)-10,19-Dihydroercalciol, (also termed dihydrotachysterol₂, hytakerol, and dihydrotachysterol), (24S)-Ethylcalciol (also termed vitamin D₅) and (22E)-(24R)-Ethyl-22,23-didehydrocalciol, (also termed vitamin D₆).

In one or more embodiments, the steroid is a phytosteroid or a phytosterol. As used herein, the term “phytosteroid” or “phytosterol” includes all steroids that are obtained, derived or extracted from plant sources. Non-limiting examples of families of phytosteroids and phytosterols include ecdysones, withanolids, sterines, steroid saponins and soflavonoids. Non-limiting examples of phytosteroid and phytosterol compounds include alpha-sitosterol, beta-sitosterol, stigmastanol, campesterol, alpha-sitostanol, beta-sitostanol, stigmastanol, campestanol, avenosterol, brassicasterol, desmosterol, chalinosterol, beta-ecdysone, whithaferin A, beta-sitosterine, stigmasterine, campesterine, ergosterine, diosgenin, daidzein, glycitein, genistein, muristerone, poriferasterol, clionasterol, campestanol, and cycloartenol, as well as all natural or synthesized forms and derivatives thereof, such as fatty acid esters, such as ferulic acid esters, oleoyl esters, and cinnamic acid esters, including isomers.

Plant oils and extracts which contain steroids are also useful. Non limiting examples of plants that contain steroids include nuts seeds, sprouted seeds and grains (such as alfalfa), St. Mary's thistle, ginkgo biloba, saw palmetto, panax, siberian ginseng, foeniculum vulgare, cimicifuga racemosa, licorice root, red clover, sage, sarsaparilla, sassafras, angelica sinensis achillea millefolium, anemone pratensis, angelica sinensis, glycyrrhiza glabra, hypericum perforatum, larrea, panax, piscidia erythrina, plantago psyllium, serenoa repens, symphytum, taraxacum officinale, trifolium pratense, tumera spp., tussilago farfara, valeriana officinalis, viburnum prunifolium, calendula officinalis

In one or more embodiments, the steroid is a compound that is positively identified using a laboratory method, suitable of detecting a steroid.

Several disorders of the skin, a body cavity or mucosal surface (e.g., the mucosa of the nose, mouth, eye, ear, vagina or rectum) involve a combination of inflammation, cell proliferation and differentiation abnormalities, and other biological abnormalities that can be effected by a steroid; and other etiological factors that require an additional therapeutic modality. For example, psoriasis involves inflammation as well as excessive cell proliferation and inadequate cell differentiation. Atopic dermatitis involves inflammation, skin dryness and keratinocyte growth abnormality. Bacterial, fungal and viral infections involve pathogen colonization at the affected site and inflammation. Likewise, hair growth disorders and other pilosebaceous disorders involve an impaired hormonal balance (which can be affected by a steroid hormone or a steroid hormone antagonist), together with other etiological factors, that can be affected a non-steroidal active agent. Hence, in many cases, the inclusion of an additional therapeutic agent in the foamable pharmaceutical composition of the present invention, contributes to the clinical activity of the steroid. Thus, in one or more embodiments, the foamable composition further includes at least one additional therapeutic agent, in a therapeutically effective concentration.

In one or more embodiments, the at least one additional therapeutic agent is selected from the group consisting of an anti-infective, an antibiotic, an antibacterial agent, an antifungal agent, an antiviral agent, an antiparasitic agent, a nonsteroidal anti-inflammatory drug, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, an anti-wrinkle agent, a radical scavenger, a metal oxide (e.g., titanium dioxide, zinc oxide, zirconium oxide, iron oxide), silicone oxide, an anti wrinkle agent, a skin whitening agent, a skin protective agent, a masking agent, an anti-wart agent, a refatting agent, a lubricating agent and mixtures thereof.

In certain cases, the disorder to be treated involves unaesthetic lesions that need to be masked. For example, rosacea involves papules and pustules, which can be treated with a steroid, as well as erythema, telangiectasia and redness, which do not respond to treatment with a steroid. Thus, in one or more embodiments, the additional active agent is a masking agent, i.e., a pigment. Non limiting examples of suitable pigments include brown, yellow or red iron oxide or hydroxides, chromium oxides or hydroxides, titanium oxides or hydroxides, zinc oxide, FD&C Blue No. 1 aluminum lake, FD&C Blue No. 2 aluminum lake and FD&C Yellow No. 6 aluminum lake.

The foamable composition of the present invention can be an emulsion, or microemulsion, including an aqueous phase and an organic carrier phase. The organic carrier is selected from a hydrophobic organic carrier (also termed herein “hydrophobic solvent”), an emollient, a polar solvent, and a mixture thereof.

A “hydrophobic organic carrier” as used herein refers to a material having solubility in distilled water at ambient temperature of less than about 1 gm per 100 mL, more preferable less than about 0.5 gm per 100 mL, and most preferably less than about 0.1 gm per 100 mL. It is liquid at ambient temperature. The identification of a hydrophobic organic carrier or “hydrophobic solvent”, as used herein, is not intended to characterize the solubilization capabilities of the solvent for any specific active agent or any other component of the foamable composition. Rather, such information is provided to aid in the identification of materials suitable for use as a hydrophobic carrier in the foamable compositions described herein.

In one or more embodiments, the hydrophobic organic carrier is an oil, such as mineral oil. Mineral oil (Chemical Abstracts Service Registry number 8012-95-1) is a mixture of aliphatic, naphthalenic, and aromatic liquid hydrocarbons that derive from petroleum. It is typically liquid; its viscosity is in the range of between about 35 CST and about 100 CST (at 40° C.), and its pour point (the lowest temperature at which an oil can be handled without excessive amounts of wax crystals forming so preventing flow) is below 0° C. Term hydrophobic organic carrier does not include thick or semi-solid materials, such as white petrolatum, also termed “Vaseline”, which, in certain compositions is disadvantageous due to its waxy nature and semi-solid texture.

According to one or more embodiments, hydrophobic solvents are liquid oils originating from vegetable, marine or animal sources. Suitable liquid oil includes saturated, unsaturated or polyunsaturated oils. By way of example, the unsaturated oil may be olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils or mixtures thereof, in any proportion.

Suitable hydrophobic solvents also include polyunsaturated oils containing poly-unsaturated fatty acids. In one or more embodiments, the unsaturated fatty acids are selected from the group of omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Such unsaturated fatty acids are known for their skin-conditioning effect, which contribute to the therapeutic benefit of the present foamable composition. Thus, the hydrophobic solvent can include at least 6% of an oil selected from omega-3 oil, omega-6 oil, and mixtures thereof. In the context of the present invention, oils that possess therapeutically-beneficial properties are termed “therapeutically active oil”.

Another class of hydrophobic solvents is the essential oils, which are also considered therapeutically active oil, which contain active biologically occurring molecules and, upon topical application, exert a therapeutic effect, which is conceivably synergistic to the beneficial effect of the steroid in the composition.

Another class of therapeutically active oils includes liquid hydrophobic plant-derived oils, which are known to possess therapeutic benefits when applied topically.

Silicone oils also may be used and are desirable due to their known skin protective and occlusive properties. Suitable silicone oils include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These are chosen from cyclic or linear polydimethylsiloxanes containing from about 3 to about 9, preferably from about 4 to about 5, silicon atoms. Volatile silicones such as cyclomethicones can also be used. Silicone oils are also considered therapeutically active oil, due to their barrier retaining and protective properties.

In one or more embodiments, the hydrophobic carrier includes at least 2% by weight silicone oil or at least 5% by weight.

The solvent may be a mixture of two or more of the above hydrophobic solvents in any proportion.

A further class of solvents includes “emollients” that have a softening or soothing effect, especially when applied to body areas, such as the skin and mucosal surfaces. Emollients are not necessarily hydrophobic. Examples of suitable emollients include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof.

According to one or more embodiments of the present invention, the hydrophobic organic carrier includes a mixture of a hydrophobic solvent and an emollient. According to one or more embodiments, the foamable composition is a mixture of mineral oil and an emollient in a ratio between 2:8 and 8:2 on a weight basis.

A “polar solvent” is an organic solvent, typically soluble in both water and oil. Examples of polar solvents include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, sulfoxides, such as dimethylsulfoxide (DMSO), dimethylformamide, methyl dodecyl sulfoxide, dimethylacetamide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

The polymeric agent serves to stabilize the foam composition and to control drug residence in the target organ. Exemplary polymeric agents, are classified below in a non-limiting manner. In certain cases, a given polymer can belong to more than one of the classes provided below.

In one or more embodiments, the composition of the present invention includes at least one gelling agent. A gelling agent controls the residence of a therapeutic composition in the target site of treatment by increasing the viscosity of the composition, thereby limiting the rate of its clearance from the site. Many gelling agents are known in the art to possess mucoadhesive properties.

The gelling agent can be a natural gelling agent, a synthetic gelling agent and an inorganic gelling agent. Exemplary gelling agents that can be used in accordance with one or more embodiments of the present invention include, for example, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), guar gum, hydroxypropyl guar gum, soluble starch, cationic celluloses, cationic guars, and the like, and synthetic polymeric materials, such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like. Mixtures of the above compounds are contemplated.

Further exemplary gelling agents include the acrylic acid/ethyl acrylate copolymers and the carboxyvinyl polymers sold, for example, by the B.F. Goodrich Company under the trademark of Carbopol® resins. These resins consist essentially of a colloidal water-soluble polyalkenyl polyether crosslinked polymer of acrylic acid crosslinked with from 0.75% to 2% of a crosslinking agent such as polyallyl sucrose or polyallyl pentaerythritol. Examples include Carbopol® 934, Carbopol® 940, Carbopol® 950, Carbopol® 980, Carbopol® 951 and Carbopol® 981. Carbopol® 934 is a water-soluble polymer of acrylic acid crosslinked with about 1% of a polyallyl ether of sucrose having an average of about 5.8 allyl groups for each sucrose molecule.

In one or more embodiment, the composition of the present invention includes at least one polymeric agent, which is a water-soluble cellulose ether. Preferably, the water-soluble cellulose ether is selected from the group consisting of methylcellulose, hydroxypropyl cellulose, hydroxypropyl methylcellulose (Methocel), hydroxyethyl cellulose, methylhydroxyethylcellulose, methylhydroxypropylcellulose, hydroxyethylcarboxymethylcellulose, carboxymethylcellulose and carboxymethylhydroxyethylcellulose. More preferably, the water-soluble cellulose ether is selected from the group consisting of methylcellulose, hydroxypropyl cellulose and hydroxypropyl methylcellulose (Methocel). In one or more embodiments, the composition includes a combination of a water-soluble cellulose ether; and a naturally-occurring polymeric materials, selected from the group including xanthan gum, guar gum, carrageenan gum, locust bean gum and tragacanth gum.

Yet, in other embodiments, the gelling agent includes inorganic gelling agents, such as silicone dioxide (fumed silica).

Mucoadhesive/bioadhesion has been defined as the attachment of synthetic or biological macromolecules to a biological tissue. Mucoadhesive agents are a class of polymeric biomaterials that exhibit the basic characteristic of a hydrogel, i.e. swell by absorbing water and interacting by means of adhesion with the mucous that covers epithelia. Compositions of the present invention may contain a mucoadhesive macromolecule or polymer in an amount sufficient to confer bioadhesive properties. The bioadhesive macromolecule enhances the delivery of biologically active agents on or through the target surface. The mucoadhesive macromolecule may be selected from acidic synthetic polymers, preferably having at least one acidic group per four repeating or monomeric subunit moieties, such as poly(acrylic)- and/or poly(methacrylic) acid (e.g., Carbopol®, Carbomer®), poly(methylvinyl ether/maleic anhydride) copolymer, and their mixtures and copolymers; acidic synthetically modified natural polymers, such as carboxymethylcellulose (CMC); neutral synthetically modified natural polymers, such as (hydroxypropyl)methylcellulose; basic amine-bearing polymers such as chitosan; acidic polymers obtainable from natural sources, such as alginic acid, hyaluronic acid, pectin, gum tragacanth, and karaya gum; and neutral synthetic polymers, such as polyvinyl alcohol or their mixtures. An additional group of mucoadhesive polymers includes natural and chemically modified cyclodextrin, especially hydroxypropyl-β-cyclodextrin. Such polymers may be present as free acids, bases, or salts, usually in a final concentration of about 0.01% to about 0.5% by weight.

A suitable bioadhesive macromolecule is the family of acrylic acid polymers and copolymers, (e.g., Carbopol®). These polymers contain the general structure —[CH₂—CH(COOH)—]ₙ. Hyaluronic acid and other biologically-derived polymers may be used.

Exemplary bioadhesive or mucoadhesive macromolecules have a molecular weight of at least 50 kDa, or at least 300 kDa, or at least 1,000 kDa. Favored polymeric ionizable macromolecules have not less than 2 mole percent acidic groups (e.g., COOH, SO3H) or basic groups (NH2, NRH, NR2), relative to the number of monomeric units. The acidic or basic groups can constitute at least 5 mole percent, or at least 10 mole percent, or at least 25, at least 50 more percent, or even up to 100 mole percent relative to the number of monomeric units of the macromolecule.

Yet, another group of mucoadhesive agent includes inorganic gelling agents such as silicon dioxide (fumed silica), including but not limited to, AEROSIL 200 (DEGUSSA).

Many mucoadhesive agents are known in the art to also possess gelling properties.

The foam composition may contain a film forming component. The film forming component may include at least one water-insoluble alkyl cellulose or hydroxyalkyl cellulose. Exemplary alkyl cellulose or hydroxyalkyl cellulose polymers include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross linking agent may be used to modify the polymer's characteristics. For example, esters such as dibutyl or diethyl phthalate, amides such as diethyldiphenyl urea, vegetable oils, fatty acids and alcohols such as oleic and myristyl acid may be used in combination with the cellulose derivative.

In one or more embodiments, the composition of the present invention includes a phase change polymer, which alters the composition behavior from fluid-like prior to administration to solid-like upon contact with the target mucosal surface. Such phase change results from external stimuli, such as changes in temperature or pH and exposure to specific ions (e.g., Ca²⁺).

Non-limiting examples of phase change polymers include poly(N-isopropylamide) and Poloxamer 407®.

The polymeric agent is present in an amount in the range of about 0.01% to about 5.0% by weight of the foam composition. In one or more embodiments, it is typically less than about 1 wt % of the foamable composition.

Surface-active agents (also termed “surfactants”) include any agent linking oil and water in the composition, in the form of emulsion. A surfactant's hydrophilic/lipophilic balance (HLB) describes the emulsifier's affinity toward water or oil. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average).

According to one or more embodiments of the present invention, the surface-active agent has a hydrophilic lipophilic balance (HLB) between about 9 and about 14, which is the required HLB (the HLB required to stabilize an O/W emulsion of a given oil) of most oils and hydrophobic solvents. Thus, in one or more embodiments, the composition contains a single surface active agent having an HLB value between about 9 and 14, and in one or more embodiments, the composition contains more than one surface active agent and the weighted average of their HLB values is between about 9 and about 14. Yet, in other embodiments, when a water in oil emulsion is desirable, the composition contains one or more surface active agents, having an HLB value between about 2 and about 9.

The surface-active agent is selected from anionic, cationic, nonionic, zwitterionic, amphoteric and ampholytic surfactants, as well as mixtures of these surfactants. Such surfactants are well known to those skilled in the therapeutic and cosmetic formulation art. Nonlimiting examples of possible surfactants include polysorbates, such as polyoxyethylene (20) sorbitan monostearate (Tween 60) and poly(oxyethylene) (20) sorbitan monooleate (Tween 80); poly(oxyethylene) (POE) fatty acid esters, such as Myrj 45, Myrj 49, Myrj 52 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; mono or diglycerides, isoceteth-20, sodium methyl cocoyl taurate, sodium methyl oleoyl taurate, sodium lauryl sulfate, triethanolamine lauryl sulfate and betaines.

In one or more embodiments of the present invention, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. We have surprisingly found that non-ionic surfactants alone provide foams of excellent quality, i.e. a score of “E” according to the grading scale discussed herein below.

In one or more embodiments, the surface active agent includes a mixture of at least one non-ionic surfactant and at least one ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1.

In one or more embodiments of the present invention, a combination of a non-ionic surfactant and an ionic surfactant (such as sodium lauryl sulphate and cocamidopropylbetaine) is employed, at a ratio of between 1:1 and 20:1, or at a ratio of 4:1 to 10:1. The resultant foam has a low specific gravity, e.g., less than 0.1 g/ml.

It has been surprisingly discovered that the stability of the composition is especially pronounced when a combination of at least one non-ionic surfactant having HLB of less than 9 and at least one non-ionic surfactant having HLB of equal or more than 9 is employed. The ratio between the at least one non-ionic surfactant having HLB of less than 9 and the at least one non-ionic surfactant having HLB of equal or more than 9, is between 1:8 and 8:1, or at a ratio of 4:1 to 1:4. The resultant HLB of such a blend of at least two emulsifiers is between about 9 and about 14.

Thus, in an exemplary embodiment, a combination of at least one non-ionic surfactant having HLB of less than 9 and at least one non-ionic surfactant having HLB of equal or more than 9 is employed, at a ratio of between 1:8 and 8:1, or at a ratio of 4:1 to 1:4, wherein the HLB of the combination of emulsifiers is between about 9 and about 14.

In one or more embodiments of the present invention, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

The total surface active agent is in the range of about 0.1 to about 5% of the foamable composition, and is typically less than about 2% or less than about 1%.

Preferably a therapeutically effective foam adjuvant is included in the foamable compositions of the present invention to increase the foaming capacity of surfactants and/or to stabilize the foam. In one or more embodiments of the present invention, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of the fatty alcohol required to support the foam system is inversely related to the length of its carbon chains. Foam adjuvants, as defined herein are also useful in facilitating improved spreadability and absorption of the composition.

In one or more embodiments of the present invention, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

In one or more embodiments, a combination of a fatty acid and a fatty ester is employed.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

An important property of the fatty alcohols and fatty acids used in context of the composition of the present invention is related to their therapeutic properties per se. Long chain saturated and mono unsaturated fatty alcohols, e.g., stearyl alcohol, erucyl alcohol, arachidyl alcohol and behenyl alcohol (docosanol) have been reported to possess antiviral, antiinfective, antiproliferative and antiinflammatory properties (see, U.S. Pat. No. 4,874,794). Longer chain fatty alcohols, e.g., tetracosanol, hexacosanol, heptacosanol, octacosanol, triacontanol, etc., are also known for their metabolism modifying properties and tissue energizing properties. Long chain fatty acids have also been reported to possess anti-infective characteristics.

Thus, in preferred embodiments of the present invention, a combined and enhanced therapeutic effect is attained by including both a steroid and a therapeutically effective foam adjuvant in the same composition, thus providing a simultaneous anti-inflammatory and antiinfective effect from both components. Furthermore, in a further preferred embodiment, the composition concurrently comprises a steroid, a therapeutically effective foam adjuvant and a therapeutically active oil, as detailed above. Such combination provides an even more enhanced therapeutic benefit. Thus, the foamable carrier, containing the foam adjuvant provides an extra therapeutic benefit in comparison with currently used vehicles, which are inert and non-active.

The foam adjuvant according to one or more preferred embodiments of the present invention includes a mixture of fatty alcohols, fatty acids and hydroxy fatty acids and derivatives thereof in any proportion, providing that the total amount is 0.1% to 5% (w/w) of the carrier mass. More preferably, the total amount is 0.4%-2.5% (w/w) of the carrier mass.

The therapeutic foam of the present invention may further optionally include a variety of formulation excipients, which are added in order to fine-tune the consistency of the formulation, protect the formulation components from degradation and oxidation and modify their consistency. Such excipients may be selected, for example, from stabilizing agents, antioxidants, humectants, preservatives, colorant and odorant agents and other formulation components, used in the art of formulation.

Optionally, the composition further contains a penetration enhancer. This is particularly important when the steroid is supposed to reach deeper layers of the target tissue or when the active agent is intended for systemic administration, via the transdermal or transmucosal route. Non limiting examples of penetration enhancers include propylene glycol, butylene glycols, glycerol, pentaerythritol, sorbitol, mannitol, oligosaccharides, dimethyl isosorbide, monooleate of ethoxylated glycerides having about 8 to 10 ethylene oxide units, polyethylene glycol 200-600, transcutol, glycofurol and cyclodextrins.

Aerosol propellants are used to generate and administer the foamable composition as a foam. The total composition including propellant, foamable compositions and optional ingredients is referred to as the foamable carrier. The propellant makes up about 3% to about 25 wt % of the foamable carrier. Examples of suitable propellants include volatile hydrocarbons such as butane, propane, isobutane or mixtures thereof, and fluorocarbon gases.

Composition and Foam Physical Characteristics

A pharmaceutical or cosmetic composition manufactured using the foam carrier according to one or more embodiments of the present invention is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foam composition of the present invention creates a stable emulsion having an acceptable shelf-life of at least one year, or at least two years at ambient temperature. A feature of a product for cosmetic or medical use is long term stability. Propellants, which are a mixture of low molecular weight hydrocarbons, tend to impair the stability of emulsions. It has been observed, however, that emulsion foam compositions according to the present invention are surprisingly stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam. Compositions containing semi-solid hydrophobic solvents, e.g., white petrolatum, as the main ingredients of the oil phase of the emulsion, exhibit high viscosity and poor flowability and are inappropriate candidates for a foamable composition.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

As further aspect of the foam is breakability. The breakable foam is thermally stable, yet breaks under sheer force. Sheer-force breakability of the foam is clearly advantageous over thermally-induced breakability. Thermally sensitive foams immediately collapse upon exposure to skin temperature and, therefore, cannot be applied on the hand and afterwards delivered to the afflicted area.

Another property of the foam is specific gravity, as measured upon release from the aerosol can. Typically, foams have specific gravity of less than 0.1 g/mL or less than 0.05 g/mL.

Fields of Pharmaceutical Applications

By including an appropriate steroid and optional active agents in the compositions of the present invention, the composition are useful in treating a patient having any one of a variety of dermatological disorders, which include inflammation as one or their etiological factors (also termed “dermatoses”), such as classified in a non-limiting exemplary manner according to the following groups:
Dermatitis including contact dermatitis, atopic dermatitis, seborrheic dermatitis, nummular dermatitis, chronic dermatitis of the hands and feet, generalized exfoliative dermatitis, stasis dermatitis; lichen simplex chronicus; diaper rash;
Bacterial infections including cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma;
Fungal Infections including dermatophyte infections, yeast Infections; parasitic Infections including scabies, pediculosis, creeping eruption;
Viral Infections;
Disorders of hair follicles and sebaceous glands including acne, rosacea, perioral dermatitis, hypertrichosis (hirsutism), alopecia, including male pattern baldness, alopecia greata, alopecia universalis and alopecia totalis; pseudofolliculitis barbae, keratinous cyst;
Scaling papular diseases including psoriasis, pityriasis rosea, lichen planus, pityriasis rubra pilaris;
Benign tumors including moles, dysplastic nevi, skin tags, lipomas, angiomas, pyogenic granuloma, seborrheic keratoses, dermatofibroma, keratoacanthoma, keloid;
Malignant tumors including basal cell carcinoma, squamous cell carcinoma, malignant melanoma, paget's disease of the nipples, kaposi's sarcoma;
Reactions to sunlight, including sunburn, chronic effects of sunlight, photosensitivity;
Bullous diseases including pemphigus, bullous pemphigoid, dermatitis herpetiformis, linear immunoglobulin A disease;
Pigmentation disorders including hypopigmentation such as vitiligo, albinism and postinflammatory hypopigmentation and hyperpigmentation such as melasma (chloasma), drug-induced hyperpigmentation, postinflammatory hyperpigmentation;
Disorders of cornification including ichthyosis, keratosis pilaris, calluses and corns, actinic keratosis;
Pressure sores;
Disorders of sweating; and
Inflammatory reactions including drug eruptions, toxic epidermal necrolysis; erythema multiforme, erythema nodosum, granuloma annulare.

The same advantage is expected when the composition, including a steroid, is topically applied to a body cavity or mucosal surfaces, including, but not limited to the cranial cavity, the thoratic cavity, the abdominal cavity, the venteral cavity, the vagina, the rectum and penile cavities, the urinary tract, the nasal cavity, the mouth, the eye, the ear the peritoneum, the large and small bowel, the caecum, bladder, and stomach, the cavity between the uterus and the fallopian tubes, the ovaries and other body areas, which may accept topically-applied products. The composition of the present invention is suitable to treat conditions of a body cavity and a mucosal membrane, such as post-surgical adhesions, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

According to one or more embodiments of the present invention, the compositions are also useful in the therapy of non-dermatological disorders by providing transdermal delivery of a steroid that is effective against non-dermatological disorders.

In one or more embodiments, the disorder is a health abnormality that responds to treatment with a steroid hormone. A typical example of such abnormality is sexual dysfunction in men and women whereby androgen therapy is successfully used to restore sexual function. Other non-limiting examples of disorders/medical indications that are in the scope of treatment with a steroid hormone according to the present invention are androgen deficiency, estrogen deficiency, growth disorders, hypogonadism, cancer, vasomotor symptoms, menopausal disorders, vulvar and vaginal atrophy, urethritis, hypoestrogenism, osteoarthritis, osteoporosis, uterine bleeding, Hirsutism, Virilization, ovarian tumors, hypothalamic pituitary unit diseases, testicular tumors, prostate cancer, hypopituitarism, Klinefelter's syndrome, testicular feminisation, orchitectomy, vasomotor symptoms (such as “hot flashes”) associated with the menopause, metabolic abnormalities and mood disturbances.

The following examples exemplify the therapeutic kits and pharmacological compositions and methods described herein. The examples are for the purposes of illustration only and are not intended to be limiting of the invention.

### EXAMPLE 1

### Oil in Water Foamable Emulsion Compositions (˜12% and ˜30% Oil) Comprising Steroids with and without an Additional Active Agent

| | Composition No: | | | |
|---|---|---|---|---|
| | HP1 | HP2 | HP3 | HB1 |
| | % | | | |
| Ingredient | | | | |
| Hydrocortisone propionate | 1.00 | 1.00 | 1.00 | — |
| (Steroid) | | | | |
| Halobetasol (steroid) | — | — | — | 0.05 |
| Terbinafine (additional | — | — | 2.00 | — |
| active agent) | | | | |
| Hammamelis extract | 10.00 | — | — | 10.00 |
| (additional active agent) | | | | |
| Mineral oil | 5.60 | 5.60 | 20.00 | 5.60 |
| Isopropyl myristate | 5.60 | 5.60 | 9.00 | 5.60 |
| Glyceryl monostearate | 0.45 | 0.45 | 0.45 | 0.45 |
| Stearyl alcohol | 0.85 | 0.85 | 0.80 | 0.85 |
| Xanthan gum | 0.20 | 0.26 | 0.20 | 0.20 |
| Methocel K100M | 0.20 | 0.26 | 0.20 | 0.20 |
| Polysorbate 80 | 0.90 | 0.90 | 0.90 | 0.90 |
| PEG-40 stearate | 2.60 | 2.60 | 2.60 | 2.60 |
| EDTA disodium | 0.20 | 0.20 | — | 0.20 |
| Preservative | 0.25 | 0.25 | 0.25 | 0.25 |
| Propellant | 10.00 | 10.00 | 10.00 | 10.00 |
| Water | To 100 | To 100 | To 100 | To 100 |
| Product Characteristics | | | | |
| Emulsion uniformity | | uniform | uniform | uniform |
| Color | Brown | White | White | Brown |
| pH | | 4.73 | 5.02 | 4.73 |
| Foam quality | | E | E | E |
| Density | | 0.021 | 0.032 | 0.022 |

As shown above, Compositions HP2, HP3 and HB1 were further examined for emulsion uniformity, emulsion stability, foam quality and density and found stable, and meeting the requirements of density between 0.01-0.1 and 1 g/mL and excellent (E) quality.

### EXAMPLE 2

### Oil in Water Foamable Emulsion Compositions (˜12% and 30% Oil) Comprising Testosterone with and without an Additional Active Agent

| | Composition No: | | | |
|---|---|---|---|---|
| | TS1 | TS2 | TS3 | TS4 |
| | % | | | |
| Ingredient | | | | |
| Testosterone propionate (steroid) | 0.25 | 0.25 | 0.25 | 0.25 |
| Propylene glycol (penetration | 2.00 | 4.00 | — | — |
| enhancer) | | | | |
| Diethylene glycol monoethyl | — | — | — | 6.00 |
| ether (penetration enhancer) | | | | |
| Mineral oil | 5.60 | 5.60 | 20.00 | 20.00 |
| Isopropyl myristate | 5.60 | 5.60 | 9.00 | 9.00 |
| Glyceryl monostearate | 0.45 | 0.45 | 0.45 | 0.45 |
| Stearyl alcohol | 0.85 | 0.85 | 0.80 | 0.80 |
| Xanthan gum | 0.20 | 0.26 | 0.20 | 0.20 |
| Methocel K100M | 0.20 | 0.26 | 0.20 | 0.20 |
| Polysorbate 80 | 0.90 | 0.90 | 0.90 | 0.90 |
| PEG-40 stearate | 2.60 | 2.60 | 2.60 | 2.60 |
| EDTA disodium | 0.20 | 0.20 | — | — |
| Preservative | 0.25 | 0.25 | 0.25 | 0.25 |
| Propellant | 10.00 | 10.00 | 10.00 | 10.00 |
| Water | To 100 | To 100 | To 100 | To 100 |
| Product Characteristics | | | | |
| Emulsion uniformity | | | Uniform | |
| Color | | | White | |
| pH 1:5-foam | | | 5.02 | |
| Foam quality | | | E | |
| Density | | | 0.039 | |

As shown above, Composition TS3 was further examined for emulsion uniformity, emulsion stability, foam quality and density and found stable, and meeting the requirements of density between 0.1 and 1 g/mL and excellent (E) quality. In one or more embodiments, when the hormone composition is intended for transdermal or transmucosal administration of testosterone, the compositions can be contained in an aerosol kin, which includes a metered dose device, for accurate dosing of the active agent.

### EXAMPLE 3

### Oil in Water Anti-Inflammatory Steroid Foamable Emulsion Compositions with/without Additional Active Agents

| | Composition Code: | | | | |
|---|---|---|---|---|---|
| | HB1 | BV1 | BV2 | HB2 | HB3 |
| Ingredient | % | | | | |
| Hydrocortisone butyrate (steroid) | 0.10 | | | | |
| Betamethasone valerate (steroid) | | 0.12 | 0.12 | | |
| Haolbetasol (steroid) | | | | 0.02 | 0.01 |
| Alpha-Bisabolol (additional active agent) | | 0.20 | | 0.20 | |
| D-Panthenol 50P (additional active agent) | | 10.00 | | 10.00 | |
| Mineral oil | 12.00 | 12.00 | 12.00 | 12.00 | 12.00 |
| Isopropyl myristate | 12.00 | 12.00 | 12.00 | 12.00 | 12.00 |
| Dimeticone V100 | 3.00 | 3.00 | 3.00 | 3.00 | 3.00 |
| Glyceryl monostearate | 0.50 | 0.50 | 0.50 | 0.50 | 0.50 |
| MYRJ 52 | 3.00 | 3.00 | 3.00 | 3.00 | 3.00 |
| Microcrystalline cellulose + carboxymethyl cellulose) | 2.00 | 1.00 | 2.00 | 2.00 | 2.00 |
| TWEEN 80 | 1.00 | 1.00 | 1.00 | 1.00 | 1.00 |
| Cocoamidopropyl betaine | 0.50 | 0.50 | 0.50 | 0.50 | 0.50 |
| Preservative | 0.30 | 0.30 | 0.30 | 0.30 | 0.30 |
| Purified water | To 100 | To 100 | To 100 | To 100 | To 100 |

### EXAMPLE 4

### Oil in Water Steroid Hormone Foamable Emulsion Compositions

| | Composition Code: | | | |
|---|---|---|---|---|
| | HB1 | BV1 | BV2 | HB2 |
| Ingredient | % | | | |
| Testosterone propionate | 0.25 | 0.25 | 0.25 | 0.25 |
| (steroid hormone) | | | | |
| Propylene glycol | 2.00 | 4.00 | | |
| (penetration enhancer) | | | | |
| Diethylene glycol monoethyl ether | | | 6.00 | |
| (penetration enhancer) | | | | |
| Mineral oil | 12.00 | 12.00 | 12.00 | 12.00 |
| Isopropyl myristate | 12.00 | 12.00 | 12.00 | 12.00 |
| Dimeticone V100 | 3.00 | 3.00 | 3.00 | 3.00 |
| Glyceryl monostearate | 0.50 | 0.50 | 0.50 | 0.50 |
| MYRJ 52 | 3.00 | 3.00 | 3.00 | 3.00 |
| Microcrystalline cellulose + | 2.00 | 1.00 | 2.00 | 2.00 |
| carboxymethyl cellulose) | | | | |
| TWEEN 80 | 1.00 | 1.00 | 1.00 | 1.00 |
| Cocoamidopropyl betaine | 0.50 | 0.50 | 0.50 | 0.50 |
| Preservative | 0.30 | 0.30 | 0.30 | 0.30 |
| Purified water | To 100 | To 100 | To 100 | To 100 |

### EXAMPLE 6

### Oil in Water Foamable Emulsion Compositions Comprising Steroids from Natural Sources

Avocado (Persea gratissima) oil and extracts are used as an example of plants that contain phytosteroids. The oil and extracts contain two important phytosteroids—stigmasterol and beta-sitosterol, which are known to provide a variety of beneficial effects, such as tissue regeneration, improvement of skin elasticity, moisturization, stimulation of collagen synthesis, inhibition of leukotriene and prostaglandin formation (anti-inflammatory effect), anti-bacterial effects, skin barrier repair, film-forming properties, improvement of trans-epidermal water loss. Lanolin oil contains high levels of cholesterol, iso-cholesterol and derivatives thereof. The following compositions include avocado oil and lanolin oil as part of the oil phase of the composition.

| | Composition No: | | |
|---|---|---|---|
| | LN1 | LN2 | AV1 |
| Ingredient | % | | |
| Lanolin oil | 2.50 | 2.50 | — |
| Avocado concentrate - Avocadin, | — | — | 5.60 |
| produced by Crodarom (steroid) | | | |
| Mineral oil | 5.60 | 5.60 | — |
| Isopropyl myristate | 5.60 | 5.60 | 5.60 |
| Glyceryl monostearate | 0.45 | 0.45 | 0.45 |
| Stearyl alcohol | 0.85 | 0.85 | 0.85 |
| Xanthan gum | 0.20 | 0.20 | 0.20 |
| Methocel K100M | 0.20 | 0.20 | 0.20 |
| Polysorbate 80 | 0.90 | 0.90 | 0.90 |
| PEG-40 stearate | 2.60 | 2.60 | 2.60 |
| EDTA disodium | 0.20 | 0.20 | 0.20 |
| Preservative | 0.25 | 0.25 | 0.25 |
| Propellant | 8.00 | 8.00 | 8.00 |
| Water | To 100 | To 100 | To 100 |

### EXAMPLE 7

### An Emulsion Composition Containing a Steroid Solubilize in the Composition, but Insoluble Both in Water and in the Oil Phase of the Composition

| | Composition No: |
|---|---|
| | BV3 |
| Ingredient | % |
| Betamethasone valerate (steroid) | 0.12 |
| Mineral oil | 30.00 |
| Glyceryl stearate | 1.00 |
| Stearyl alcohol | 1.00 |
| PEG-40 stearate | 2.50 |
| Xanthan gum | 0.26 |
| Methocel K100 | 0.26 |
| Polysorbate 60 | 1.00 |
| Citric acid | 1.30 |
| Sodium citrate | 1.10 |
| Water | 61.46 |

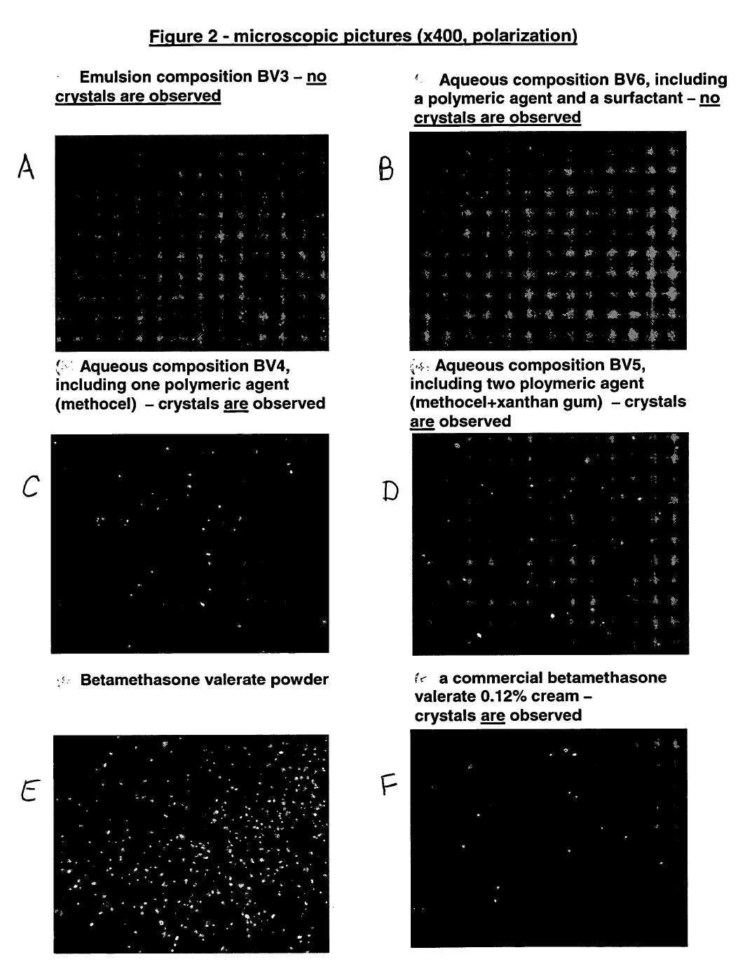

The emulsion composition of Example 7 was microscopically observed at ×400 magnification with polarization. As demonstrated in FIG. 2A (Plate 1), no crystals were observed in the emulsion composition.

In an effort to identify the components that contribute to solubilization, the aqueous phase of the emulsion was prepared, with and without a surface active agent. See, FIGS. 2B and 2C. In this particular case, the surface active agent system consisted of a combination of one non-ionic surfactants PEG-40 stearate and Polysorbate 60, as shown in the following compositions:

| | Composition No: | | |
|---|---|---|---|
| | BV4 | BV5 | BV6 |
| | Water | Water | Water |
| | Phase, | Phase, | Phase, |
| | No | With | With |
| Ingredient | Surfactant | Surfactant | Surfactant |
| Betamethasone valerate (steroid) | 0.12 gr. | 0.12 gr. | 0.12 gr. |
| PEG-40 stearate (surface | — | — | 2.50 gr. |
| active agent) | | | |
| Polysorbate 60 (surface active agent) | — | — | 1.00 gr. |
| Xanthan gum (polymeric agent) | — | 0.26 gr. | 0.26 gr. |
| Methocel K100 (polymeric agent) | 0.26 gr. | 0.26 gr. | 0.26 gr. |
| Citric acid | 1.30 gr. | 1.30 gr. | 1.30 gr. |
| Sodium citrate | 1.10 gr. | 1.10 gr. | 1.10 gr. |
| Water | 61.46 gr. | 61.46 gr. | 61.46 gr. |

As shown in FIG. 2B-2D, composition BV4 and BV5, prior to the addition of a surface active agent exhibited high crystal content (Plate 3 and 4), while in composition BV6 including a surface active agent system, no crystals were observed (Plate 2). For reference purposes in FIGS. 2E and 2F, Plates 5 and 6 illustrate the microscopic pictures of Betamethasone valerate powder and a commercial betamethasone valerate 0.12% cream (Betnovate, GlaxoSmithkline).

### EXAMPLE 8

### Comparative Study, to Assess the Organoleptic Properties of a Foamable Composition According to the Present Invention vs. a Foam Containing Petrolatum without a Foam Adjuvant and a Polymeric Agent

Usability of a pharmaceutical composition and its ease of use is a primary determinant in high treatment compliance and subsequently, favorable therapeutic results.

The vehicle of Composition HP2 (oil in water emulsion; ˜12% oil) according the Example 1 hereinabove was compared with a reference composition (Ref. Comp.) containing 10% petrolatum, 10% alkyl benzoate, 2.5% cetearyl clucoside, 72.25% water, 0.2% preservative and 5% propellant in a consumer test panel of six subjects. The panelists were asked to assess the following parameters: appearance, physical disintegration, fluidity, ease of spreading (spreadability), absorbency, residual feeling and oily feeling. As presented in the following table, the majority of panelists determined that the HP2 foam was better than Ref. Comp.

Let's Discuss

| | HP2 Better | Ref. Comp. | |
|---|---|---|---|
| | than Ref. | Better | HP2 Equals Ref. |
| | Comp. | than RA-1 | Comp. |
| Appearance | 5 | 0 | 1 |
| Physical disintegration | 4 | 1 | 1 |
| Easy to spread | 2 | 1 | 3 |
| Absorbency | 3 | 0 | 2 |
| Residual feeling | 4 | 1 | 1 |
| Oily feeling | 5 | 0 | 1 |

The multiple advantageous features of compositions HP2 are presumably attained due to the following reasons: (1) the presence of a foam adjuvant and a polymeric agent in HP2 contributes to facile spreading and absorbency; and (2) the absence of petrolatum in HP2, avoids the residual and oily feeling, typical of petrolatum-containing products. This difference is meaningful in terms of usability, compliance and consequently treatment success.

What is claimed is:

1. A therapeutic kit to provide a safe and effective dosage of a steroid, including an aerosol packaging assembly including:
a) a container accommodating a pressurized product; and
b) an outlet capable of releasing the pressurized product as a foam; wherein the pressurized product comprises a foamable composition including:
i. a steroid;
ii. at least one organic carrier selected from the group consisting of a hydrophobic organic carrier, an organic polar solvent, an emollient and mixtures thereof, at a concentration of about 2% to about 50% by weight;
iii. a surface-active agent;
iv. about 0.01% to about 5% by weight of at least one polymeric additive selected from the group consisting of a bioadhesive agent, a gelling agent, a film forming agent and a phase change agent;
v. water; and
vi. liquefied or compressed gas propellant at a concentration of about 3% to about 25% by weight of the total composition.

2. The kit of claim 1, wherein the foamable composition is selected from the group consisting of
i. an oil-in-water emulsion; and
ii. a water-in-oil emulsion.

3. The kit of claim 1, wherein the outlet comprises a valve, containing a stem with 1 to 4 apertures formed in the stem.

4. The kit of claim 3, wherein each aperture formed in the stem has a diameter, selected from the group consisting of (i) about 0.2 mm to about 1 mm; (ii) about 0.3 mm to about 0.8 mm; and (iii) about 0.01 mm² and 1 mm².

5. The kit of claim 3, wherein the sum of areas of all apertures in the stem is between about 0.04 mm² and 0.5 mm².

6. The kit of claim 3, wherein the valve is attached to metered dose device.

7. The kit of claim 1, wherein the at least one organic carrier is present in an amount selected from the group consisting of (i) about 2% to about 5%; (ii) about 5% to about 10%; (iii) about 10% to about 20%; and (iv) about 20% to about 50%.

8. The kit of claim 1, wherein the foamable composition is substantially alcohol-free.

9. The kit of claim 1, further including about 0.1% to about 5% by weight of a therapeutically active foam adjuvant is selected from the group consisting of a fatty alcohol having 15 or more carbons in their carbon chain; a fatty acid having 16 or more carbons in their carbon chain; fatty alcohols, derived from beeswax and including a mixture of alcohols, a majority of which has at least 20 carbon atoms in their carbon chain; a fatty alcohol having at least one double bond; a fatty acid having at least one double bond; a branched fatty alcohol; a branched fatty acid; a fatty acid substituted with a hydroxyl group; cetyl alcohol; stearyl alcohol; arachidyl alcohol; behenyl alcohol; 1-triacontanol; hexadecanoic acid; stearic acid; arachidic acid; behenic acid; octacosanoic acid; 12-hydroxy stearic acid and mixtures thereof.

10. The kit of claim 1, wherein the steroid is selected from the group consisting of
i a steroid compound containing a cyclopenta[a]phenanthrene skeleton;
ii a steroid compound containing a cyclopenta[a]phenanthrene skeleton carrying one or more functional groups selected from halogens, alkyl groups, aryl groups, benzyl groups, carboxy groups and alkoxy groups;
iii a steroid compound selected from the families of (a) cardanolides, (b) bufanolides, (c) spirostans, (d) furostans, (e) steroid alkaloids, (f) steroid lactones, (g) oxo-steroids, (h) steroid-alcohols and (i) steroid-amines;
iv a steroid compound, where one or more of the cyclopenta[a]phenanthrene rings is contracted by loss of an unsubstituted methylene group;
v a steroid compound, where one or more of the cyclopenta[a]phenanthrene rings is expanded by inclusion of a methylene group;
vi a steroid compound containing a cyclopenta[a]phenanthrene skeleton and a carbocyclic or heterocyclic ring component fused to it;
vii a compound, wherein two or more steroid molecules are linked together covalently;
viii a compound selected from the group consisting of 5α-pregnane, 5^{β}-pregnane, 5α-cholane (allocholane), 5^{β}-cholane, 5α-cholestane, 5^{β}-cholestane, 5α-ergostane, 5^{β}-ergostane, 5α-campestane, 5^{β}-campestane, 5α-poriferastane, 5^{β}-poriferastane, 5α-stigmastane, 5^{β}-stigmastane, 5α-gorgostaneacrihellin, actodigin, alfacalcidol, aldosterone, androsterone, betamethasone, brassinolide, calcidiol, calciol, calcitriol, canrenone, clomegestone, cholesterol, cholic acid, corticosterone, cortisol, cortisol acetate, cortisone, cortisone acetate, cyproterone, deoxycorticosterone, dexamethasone, disogluside, ecdysone, ercalciol, ergosterol, estradiol, estriol, estrone, ethinylestradiol, fluazacort, fluocortin, fusidic acid, gestrinone, gonane, halometasone, hydrocortisone, lanosterol, lithocholic acid, mebolazine, medroxyprogesterone, meproscillarin, mespirenone, mestranol, naflocort, norenthisterone, norgesterone, norgestrel, oxandrolone, oxymetholone, pancuronium bromide, prednisolone, prednisone, progesterone, proscillardin, pseudotigogenin, roxibolone, sarsasapogenin, smilagenin, spironolactone, timobesone, testosterone, tigogenin triamcinolone, ursodeoxycholic acid;
ix an anti-inflammatory steroid;
x a steroid possessing immunomodulating and/or anti-inflammatory properties;
xi a steroid, selected from the group of low-potency anti-inflammatory steroids, medium potency anti-inflammatory steroids and high potency anti-inflammatory steroids;
xii an anti-inflammatory steroid, selected from the group consisting of hydrocortisone, hydrocortisone acetate, desonide, betamethasone valerate, clobetasone-17-butyrate, flucinonide, fluocinolone acetonide, alcometasone dipropionate, mometasone furoate, prednicarbate, triamcinolone acetonide, betamethasone-17-benzoate, methylprednisolone aceponate, betamethasone dipropionate, halcinonide, triamcinolone acetonide, halobetasol, clobetasol-17-propionate;
xiii a steroid that positively affects the McKenzie vasoconstrictor assay;
xiv a steroid hormone;
xv a steroid hormone, selected from the group consisting of an androgen, an estrogen and a progestogen;
xvi an androgen, selected from the group consisting of testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone (also termed prasterone and DHEA), androstenedione, androstanediol, androsterone, androstenolone, prasterone enantate, prasterone sodium sulfate, ormeloxifene, mesterolone, fluoxymesterone, methyltestosterone, gestrinone, delmadinone, delmadinone acetate, chlormadinone, chlormadinone acetate, danazol and testolactone;
xvii an estrogen selected from the group consisting of estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate and equilin;
xviii a progestogen, selected from the group consisting of progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlormadinone acetate, dienogest, drospirenone, lynestrenol, tybolone, cyproterone acetate, megestrol acetate, nomegestrol acetate;
xix an inhibitor of a steroid hormone;
xx an inhibitor of a steroid hormone selected from the group consisting of finasteride, dutasteride and spironolactone;
xxi a vitamin D;
xxii a steroid that exhibits qualitatively the biological activity of calciol;
xxiii a vitamin D selected from the group consisting of cholecalciferol, 25-hydroxycholecalciferol, 1α,25-dihydroxycholecalciferol, ergocalciferol, 1α,25-dihydroxyergocalciferol, 22,23-dihydroergocalciferol, 1,24,25-trihydroxycholecalciferol, previtamin D₃, tachysterol₃ (also termed tacalciol);
xxiv isovitamin D₃, dihydrotachysterol₃, (1S)-hydroxycalciol, (24R)-hydroxycalcidiol, 25-fluorocalciol, ercalcidiol, ertacalciol, (5E)-isocalciol, 22,23-dihydroercalciol, (24S)-methylcalciol, (5E)-(10S)-10,19-dihydroercalciol, (24S)-ethylcalciol and (22E)-(24R)-ethyl-22,23-didehydrocalciol;
xxv a phytosteroid or a phytosterol;
xxvi a steroid derived or extracted from one of the families of phytosteroids, phytosterols, phytostanols, ecdysones, withanolids, sterines, steroid saponins and soflavonoids;
xxvii a steroid selected from the group consisting of alpha-sitosterol, beta-sitosterol, stigmastanol, campesterol, alpha-sitostanol, beta-sitostanol, stigmastanol, campestanol, avenosterol, brassicasterol, desmosterol, chalinosterol, beta-ecdysone, whithaferin A, beta-sitosterine, stigmasterine, campesterine, ergosterine, diosgenin, daidzein, glycitein, genistein, muristerone, poriferasterol, clionasterol, campestanol, and cycloartenol;
xxviii a plant oil or a plant extract, which contains a steroid;
xxix a plant oil or a plant extract, selected from the group consisting of nuts seeds, sprouted seeds and grains (such as alfalfa), St. Mary's thistle, ginkgo biloba, saw palmetto, panax, siberian ginseng, foeniculum vulgare, cimicifuga racemosa, licorice root, red clover, sage, sarsaparilla, sassafras, angelica sinensis achillea millefolium, anemone pratensis, angelica sinensis, glycyrrhiza glabra, hypericum perforatum, larrea, panax, piscidia erythrina, plantago psyllium, serenoa repens, symphytum, taraxacum officinale, trifolium pratense, turnera spp., tussilago farfara, valeriana officinalis, viburnum prunifolium, calendula officinalis;
xxx any one of the compounds exemplified in the present specification;
and salts thereof.

11. The kit of claim 1, wherein the concentration range of the steroid is selected from the group of (i) between about 0.005% and about 0.5%; (ii) between about 0.5% and about 2%; (iii) between about 2% and about 5%; and (iv) between about 5% and about 12%.

12. The kit of claim 2, wherein the graded of solubility of the steroid in the aqueous phase of the emulsion is selected from the groups consisting of:
(i) “soluble”, “freely soluble” or “very soluble”
(ii) “very slightly soluble”, “slightly soluble” or “sparingly soluble”
(iii) insoluble i.e, “requires 10,000 parts or more of a solvent to be solubilized”
where the descriptive grade of solubility is defined according to the the US Pharmacopoeia.

13. The kit of claim 2, wherein the grade of solubility of the steroid in the oil phase of the emulsion is selected from the groups consisting of:
(i) “soluble”, “freely soluble” or “very soluble”
(ii) “very slightly soluble”, “slightly soluble” or “sparingly soluble”
(iii) insoluble i.e, “requires 10,000 parts or more of a solvent to be solubilized”
where the descriptive grade of solubility is defined according to the the US Pharmacopoeia.

14. The kit of claim 1 or 2, wherein the steroid is dissolved in at least one phase of the emulsion.

15. The kit of claim 1 or 2, wherein the steroid is suspended in the composition.

16. The kit of claim 1, wherein the foamable composition further comprises at least one additional therapeutic agent selected from the group consisting of an anti-infective, an antibiotic, an antibacterial agent, an antifungal agent, an antiviral agent, an antiparasitic agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, benzoyl chloride, calcium hypochlorite, magnesium hypochlorite, an anti-wrinkle agent, a radical scavenger, a metal, silver, a metal oxide, titanium dioxide, zinc oxide, zirconium oxide, iron oxide, silicone oxide, talc, carbon, an anti wrinkle agent, a skin whitening agent, a skin protective agent, a masking agent, an anti-wart agent, a refatting agent, a lubricating agent and mixtures thereof.

17. The kit of claim 1, wherein the concentration of the surface active agent is between about 0.1% and about 5%.

18. The kit of claim 1, wherein the surface active agent includes a mixture of at least one non-ionic surfactant and at least one ionic surfactant in a ratio in the range of about 100:1 to 6:1.

19. The kit of claim 1, wherein the surface active agent comprises a combination of a non-ionic surfactant and an ionic surfactant, at a ratio of between 1:1 and 20:1.

20. The kit of claim 2, wherein the emulsion is a water in oil emulsion and wherein the HLB of the surface active agent is between about 9 and about 14.

21. The kit of claim 2, wherein the emulsion is an oil in water emulsion and wherein the HLB of the surface active agent is between about 2 and about 9.

22. The kit of claim 1, wherein the surface active agent comprises a combination of at least one non-ionic surfactant having HLB of less than 9 and at least one non-ionic surfactant having HLB of equal or more than 9, wherein the ratio between the at least one non-ionic surfactant having HLB of less than 9 and the at least one non-ionic surfactant having HLB of equal or more than 9, is between 1:8 and 8:1.

23. The kit of claim 1, wherein the polymeric agent is selected from the group consisting of a water-soluble cellulose ether and naturally-occurring polymeric material.

24. The kit of claim 23, wherein the water-soluble cellulose ether is selected from the group consisting of methylcellulose, hydroxypropyl cellulose, hydroxypropyl methylcellulose (Methocel), hydroxyethyl cellulose, methylhydroxyethylcellulose, methylhydroxypropylcellulose, hydroxyethylcarboxymethylcellulose, carboxymethylcellulose, carboxymethylhydroxyethylcellulose, xanthan gum, guar gum, carrageenin gum, locust bean gum and tragacanth gum.

25. A therapeutic foamable composition including:
i. asteroid;
ii. a therapeutically active oil;
iii. a surface-active agent;
iv. about 0.01% to about 5% by weight of at least one polymeric additive selected from the group consisting of a bioadhesive agent, a gelling agent, a film forming agent and a phase change agent;
v. water; and
vi. liquefied or compressed gas propellant at a concentration of about 3% to about 25% by weight of the total composition.
Where the composition is an emulsion.

26. The composition of claim 25, further including about 0.1% to about 5% by weight of a therapeutically active foam adjuvant is selected from the group consisting of a fatty alcohol having 15 or more carbons in their carbon chain; a fatty acid having 16 or more carbons in their carbon chain; fatty alcohols, derived from beeswax and including a mixture of alcohols, a majority of which has at least 20 carbon atoms in their carbon chain; a fatty alcohol having at least one double bond; a fatty acid having at least one double bond; a branched fatty alcohol; a branched fatty acid; a fatty acid substituted with a hydroxyl group; cetyl alcohol; stearyl alcohol; arachidyl alcohol; behenyl alcohol; 1-triacontanol; hexadecanoic acid; stearic acid; arachidic acid; behenic acid; octacosanoic acid; 12-hydroxy stearic acid and mixtures thereof.

27. The composition of claim 25 or 26, wherein the graded of solubility of the steroid in the aqueous phase of the emulsion is selected from the groups consisting of:
(i) “soluble”, “freely soluble” or “very soluble”
(ii) “very slightly soluble”, “slightly soluble” or “sparingly soluble”
(iii) insoluble i.e, “requires 10,000 parts or more of a solvent to be solubilizes”
where the descriptive grade of solubility is defined according to the US Pharmacopoeia.

28. The composition of claim 25 or 26, wherein the graded of solubility of the steroid in the oil phase of the emulsion is selected from the groups consisting of:
(i) “soluble”, “freely soluble” or “very soluble”
(ii) “very slightly soluble”, “slightly soluble” or “sparingly soluble”
(iii) insoluble i.e, “requires 10,000 parts or more of a solvent to be solubilizes”
where the descriptive grade of solubility is defined according to the US Pharmacopoeia.

29. The composition of claim 25 or 26, wherein the steroid is dissolved in at least one phase of the emulsion.

30. The composition of claim 25 or 26, wherein the steroid is suspended in the composition.

31. The composition of claim 25 or 26, wherein the foamable composition further comprises at least one additional therapeutic agent

32. The composition of claim 25, wherein the additional therapeutic agent is selected from the group consisting of an anti-infective, an antibiotic, an antibacterial agent, an antifungal agent, an antiviral agent, an antiparasitic agent, an steroidal antiinflammatory agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a steroid, an antiproliferative agent, an anticancer agent, a photodynamic therapy agent, benzoyl chloride, calcium hypochlorite, magnesium hypochlorite, an anti-wrinkle agent, a radical scavenger, a metal, silver, a metal oxide, titanium dioxide, zinc oxide, zirconium oxide, iron oxide, silicone oxide, talc, carbon, an anti wrinkle agent, a skin whitening agent, a skin protective agent, a masking agent, an anti-wart agent, a refatting agent, a lubricating agent and mixtures thereof.

33. The composition of claim 25, wherein the composition does not contain petrolatum.

34. A method of treating, alleviating or preventing a disorders of the skin, a body cavity or a mucosal surface, wherein the disorder involves inflammation as one of its etiological factors, including:
administering topically to a subject having the disorder, a foamed composition including:
a) a steroid;
b) at least one organic carrier selected from a hydrophobic organic carrier, a polar solvent, an emollient and mixtures thereof, at a concentration of about 2% to about 50% by weight;
c) about 0.1% to about 5% by weight of a surface-active agent;
d) about 0.01% to about 5% by weight of a polymeric additive selected from a bioadhesive agent, a gelling agent, a film forming agent and a phase change agent; and
e) water,
wherein the steroid is administered in a therapeutically effective amount.

35. The method of claim 34, wherein the composition further comprises about 0.1% to about 5% by weight of a therapeutically active foam adjuvant is selected from the group consisting of a fatty alcohol having 15 or more carbons in their carbon chain; a fatty acid having 16 or more carbons in their carbon chain; fatty alcohols, derived from beeswax and including a mixture of alcohols, a majority of which has at least 20 carbon atoms in their carbon chain; a fatty alcohol having at least one double bond; a fatty acid having at least one double bond; a branched fatty alcohol; a branched fatty acid; a fatty acid substituted with a hydroxyl group; cetyl alcohol; stearyl alcohol; arachidyl alcohol; behenyl alcohol; 1-triacontanol; hexadecanoic acid; stearic acid; arachidic acid; behenic acid; octacosanoic acid; 12-hydroxy stearic acid and mixtures thereof.

36. The method of claim 34, wherein the disorder is selected from the group consisting of a dermatose, a dermatitis, a vaginal disorder, a vulvar disorder, an anal disorder, a disorder of a body cavity, a disorder of the cranial cavity, the thoratic cavity, the abdominal cavity, the venteral cavity, the vagina, the rectum and penile cavities, the urinary tract, the nasal cavity, the mouth, the eye, the ear the peritoneum, the large and small bowel, the caecum, bladder, and stomach, the cavity between the uterus and the fallopian tubes, the ovaries, a disorder of the respiratory system, post-surgical adhesion, a bacterial infection, fungal infection, viral infection, dermatosis, dermatitis, parasitic infections, disorders of hair follicles and sebaceous glands, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, osteoarthritis, joint pain, hormonal disorder, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum;

37. The method of claim 34, wherein the composition is useful in the therapy of non-superficial abnormality, by providing transdermal or transmucosal delivery of a steroid that is effective against the abnormality.

38. The method of claim 37, wherein the abnormality is a disorder that responds to treatment with a steroid hormone.

39. The method of claim 34, wherein the abnormality is selected from the group consisting of a sexual dysfunction in men and women, androgen deficiency; estrogen deficiency, growth disorders, hypogonadism, cancer, vasomotor symptoms, menopausal disorders, vulvar and vaginal atrophy, urethritis, hypoestrogenism, osteoarthritis, osteoporosis, uterine bleeding, Hirsutism, Virilization, ovarian tumors, hypothalamic pituitary unit diseases, testicular tumors, prostate cancer, hypopituitarism, Klinefelter's syndrome, testicular feminisation, orchitectomy, vasomotor symptoms (such as “hot flashes”) associated with the menopause, metabolic abnormalities and mood disturbances.
and wherein the disorder is responsive to treatment with the steroid.

40. The foamable composition of claim 25 wherein the foamable composition contains at least one therapeutically active oil.

41. The foamable composition of claim 25,
wherein the foamable composition contains at least one therapeutically active oil; and
wherein the foamable composition further contains at least one therapeutically effective foam.

42. The method of claim 34, wherein the composition further comprises at least one additional therapeutic agent.

43. The foamable composition of claim 25 wherein the composition further contains a penetration enhancer.

44. A pharmaceutical composition, consisting of an oilphase and an aqueous phase, containing water and a surface active agent, having an HLB value between about 9 and about 16, and a steroid, wherein the steroid is solubilized in the composition, while it is insoluble both in water and in the oil phase of the composition.

45. The pharmaceutical composition of claim 44, wherein the steroid is selected from the group of low-potency anti-inflammatory steroids, medium potency anti-inflammatory steroids and high potency anti-inflammatory steroids.

46. The pharmaceutical composition of claim 44, further containing at least one component, selected from the group consisting of
i. at least one organic carrier selected from a hydrophobic organic carrier, a polar solvent, an emollient and mixtures thereof, at a concentration of about 2% to about 50% by weight;
ii. about 0.01% to about 5% by weight of a polymeric additive selected from a bioadhesive agent, a gelling agent, a film forming agent and a phase change agent

47. The pharmaceutical composition of claim 46, further containing a propellant, wherein the composition is contained in a pressurized container.
